SEQUENCE LISTING (7 sequences)

SEQ ID NO: 1 - protein, 157 aa, homo sapiens IL-18
YFGKLESKLSVIRNLNDQVLFIDQGNRPLFEDMTDSDCRDNAPRTIFIISMYKDSQPRGMAVTISVKCEKISTLSCENKIISFKEMNPPDNIKDTKSDIIFFQRSVPGHDNKMQFESSSYEGYFLACEKERDLFKLILKKEDELGDRSIMFTVQNED

SEQ ID NO: 2 - protein, 157 aa, murine IL-18
NFGRLHCTTAVIRNINDQVLFVDKRQPVFEDMTDIDQSASEPQTRLIIYMYKDSEVRGLAVTLSVKDSKMSTLSCKNKIISFEEMDPPENIDDIQSDLIFFQKRVPGHNKMEFESSLYEGHFLACQKEDDAFKLILKKKDENGDKSVMFTLTNLHQS

SEQ ID NO: 3 - DNA, 20 bp, Artificial Sequence
  Chemically Synthesized
tccatgacgttcctgacgtt

SEQ ID NO: 4 - DNA, 18 bp, Artificial Sequence
  Chemically Synthesized
tctcccagcgtgcgccat

SEQ ID NO: 5 - DNA, 30 bp, Artificial Sequence
  Chemically Synthesized
accgatgacgtcgccggtgacggcaccacg

SEQ ID NO: 6 - DNA, 24 bp, Artificial Sequence
  Chemically Synthesized
tcgtcgttttgtcgttttgtcgtt

SEQ ID NO: 7 - DNA, 20 bp, Artificial Sequence
  Chemically Synthesized
tccatgacgttcctgatgct

(19) United States
(12) Patent Application Publication
(10) Pub. No.: US 2007/0212329 A1
(43) Pub. Date: Sep. 13, 2007

(54) Vaccine Compositions Comprising An Interleukin 18 And Saponin Adjuvant System

(76) Inventors: Claudine Elvire Marie Bruck, King of Prussia, PA (US); Catherine Marie Ghislaine Gerard, Rixensart (BE); Zdenka Ludmila Jonak, King Of Prussia, PA (US)

Correspondence Address: SMITHKLINE BEECHAM CORPORATION;CORPORATE INTELLECTUAL PROPERTY-US, UW2220, P. O. BOX 1539, KING OF PRUSSIA, PA, 19406-0939, US

(21) Appl. No.: 10/575,838

(22) PCT Filed: Oct. 11, 2004
(86) PCT No.: PCT/EP04/11620

§ 371 Date: Oct. 16, 2006

(30) Foreign Application Priority Data
Oct. 13, 2003 (GB) 0323965.4

(51) Int. Cl. A61K 38/20; A61K 31/704; A61K 39/00; A61K 39/21; A61K 39/145; A61K 39/15; A61K 39/29; A61K 39/25; A61K 39/02; A61K 39/108; A61K 39/04; A61K 39/085; A61K 39/118; A61K 39/245; A61K 39/095; A61K 39/015

(52) U.S. Cl. 424/85.2; 424/208.1; 424/234.1; 424/228.1; 424/225.1; 424/215.1; 424/230.1; 424/209.1; 424/243.1; 424/251.1; 424/250.1; 424/241.1; 424/248.1; 424/263.1; 424/268.1; 424/269.1; 424/185.1; 514/26; 514/33

(57) ABSTRACT

This invention relates to a combination therapy that finds utility in the treatment or prophylaxis of infectious diseases, cancers, autoimmune diseases and related conditions. In particular, the combination therapy comprises the administration of a TH-1 cytokine, in particular, IL-18, and an immunogenic composition, in particular, a vaccine, comprising an antigen and a saponin adjuvant. In particular, the invention relates to the use of IL-18 or bioactive fragment or variant thereof and an immunogenic composition comprising a tumour-associated antigen and a saponin adjuvant, for the treatment of preneoplasic lesions or cancer.

### BACKGROUND OF THE INVENTION

The invention relates a combination therapy that finds utility in the treatment or prophylaxis of infectious diseases, cancers, autoimmune diseases and related conditions. In particular, the combination therapy comprises the administration of a TH-1 cytokine, in particular IL-18, and an immunogenic composition, in particular a vaccine, comprising an antigen and a saponin adjuvant. In particular the invention relates to the use of IL-18 or bioactive fragment or variant thereof and an immunogenic composition comprising a tumour-associated antigen and a saponin adjuvant, for the treatment of preneoplasic lesions or cancer. The invention further relates to combined preparations and pharmaceutical kits suitable for use according to the present invention. These methods of treatment and pharmaceutical preparations are especially useful for the stimulation of an immune response suitable for prophylactic and immunotherapeutic applications, especially for the prevention and/or treatment of tumours.

Cancer is a disease developing from a single cell due to genetic changes. Despite enormous investments of financial and human resources, cancer remains one of the major causes of death. Clinical detection of these tumours occurs mostly in a relatively late stage of disease, when the primary tumour can be removed by surgery, and the existence of micro metastases settled in different organs has often already occurred. Despite considerable advances in understanding the mechanisms leading to cancer, there has been less progress in therapy of metastatic cancers and in preventing the progression of early stages tumours towards more malignant and metastatic lesions. Chemotherapy does often not completely eliminate these cells, which then remain as a source for recurrent disease.

TH-1 type cytokines, e.g., IFN-γ, TNFα, IL-2, IL-12, IL-18, etc, tend to favor the induction of cell mediated immune responses to an administered antigen. In contrast, high levels of Th2-type cytokines (e.g., IL4, IL-5, IL-6 and IL-10) tend to favor the induction of humoral immune responses. Interleukin-18 (IL-18), also known as interferon-gamma (IFNg) inducing factor, has been described as an pleotropic cytokine with immunomodulatory effects that stimulates patient's own immune system against disease (e.g., cancer). IL-18 is expressed early in the immune response, and acts on both humoral and cellular immune responses and drives the response towards a better TH-1 type profile. It is produced by activated antigen-presenting cells and has been reported to have several bioactivities, specifically to promote the differentiation of naïve CD4 T cells into Th1 cells, to stimulate natural killer (NK) cells, natural killer T (NKT) cells, and to induce the proliferation of activated T cells, predominantly cytotoxic T cells (CD8+ phenotype) to secrete gamma interferon (IFN-gamma) (Okamura H. et al. 1998, Adv. Immunol. 70: 281-312). IL-18 also mediates Fas-induced tumor death, promotes the production of IL-1a and GMCSF, and has anti-angiogenic activity.

IL-18 has the capacity to stimulate innate immunity and both Th1- and Th2-mediated responses. In the presence of IL-12, IL-18 can act on Th1 cells, nonpolarized T cells, NK cells, B cells and dendritic cells to produce IFNg. Without IL-12 help, IL-18 has potential to induce IL-4 and IL-13 production in T cells, NK cells, mast cells and basophils.

IL-18 has been shown to induce tumour regression, through the production of IFN-gamma which is a critical component of the endogenous and cytokine-induced antitumour immune responses. Efficacy has been demonstrated in different tumour animal models (Jonak Z et. al. 2002, J. Immunother. 25, S20-S27; Akamatsu S; et al. 2002, J. Immunother. 25, S28-S34). Compositions comprising IL-18 combined with other agents have been described, in particular IL-18 in combination with chemotherapeutic agents (U.S. Pat. No. 6,582,689). IL-18 has also been described as acting as an adjuvant for vaccines (WO 99/56775; WO 03/031569).

Saponins are taught in: Lacaille-Dubois, M and Wagner H. (1996. A review of the biological and pharmacological activities of saponins. Phytomedicine vol 2 pp 363-386). Saponins are steroid or triterpene glycosides widely distributed in the plant and marine animal kingdoms. Saponins are noted for forming colloidal solutions in water which foam on shaking, and for precipitating cholesterol. When saponins are near cell membranes they create pore-like structures in the membrane which cause the membrane to burst. Haemolysis of erythrocytes is an example of this phenomenon, which is a property of certain, but not all, saponins.

Saponins are known as adjuvants in vaccines for systemic administration. The adjuvant and haemolytic activity of individual saponins has been extensively studied in the art (Lacaille-Dubois and Wagner, supra). For example, Quil A (derived from the bark of the South American tree Quillaja Saponaria Molina), and fractions thereof, are described in U.S. Pat. No. 5,057,540 and “Saponins as vaccine adjuvants”, Kensil, C. R., Crit Rev Ther Drug Carrier Syst, 1996, 12 (1-2):1-55; and EP 0 362 279 B1. Particulate structures, termed Immune Stimulating Complexes (ISCOMS), comprising Quil A or fractions thereof, have been used in the manufacture of vaccines (Morein, B., EP 0 109 942 B1). These structures have been reported to have adjuvant activity (EP 0 109 942 B1; WO 96/11711). The haemolytic saponins QS21 and QS17 (HPLC purified fractions of Quil A) have been described as potent systemic adjuvants, and the method of their production is disclosed in U.S. Pat. No.5,057,540 and EP 0 362 279 B1. Also described in these references is the use of QS7 (a non-haemolytic fraction of Quil-A) which acts as a potent adjuvant for systemic vaccines. Use of QS21 is further described in Kensil et al. (1991. J. Immunology vol 146, 431-437). Combinations of QS21 and polysorbate or cyclodextrin are also known (WO 99/10008). Particulate adjuvant systems comprising fractions of QuilA, such as QS21 and QS7 are described in WO 96/33739 and WO 96/11711.

Other saponins which have been used in systemic vaccination studies include those derived from other plant species such as Gypsophila and Saponaria (Bomford et al., Vaccine, 10(9):572-577, 1992).

Saponins are also known to have been used in mucosally applied vaccine studies, which have met with variable success in the induction of immune responses. Quil-A saponin has previously been shown to have no effect on the induction of an immune response when antigen is administered intranasally (Gizurarson et al. 1994. Vaccine Research 3, 23-29). Whilst, other authors have used this adjuvant with success (Maharaj et al., Can. J. Microbiol, 1986, 32(5):414-20; Chavali and Campbell, Immunobiology, 174(3):347-59). ISCOMs comprising Quil A saponin have been used in intragastric and intranasal vaccine formulations and exhibited adjuvant activity (McI Mowat et al., 1991, Immunology, 72, 317-322; McI Mowat and Donachie, Immunology Today, 12, 383-385). QS21, the non-toxic fraction of Quil A, has also been described as an oral or intranasal adjuvant (Sumino et al., J. Virol., 1998, 72(6):4931-9; WO 98/56415).

The present invention relates to the surprising finding that combined administration of a TH-1 cytokine such as IL-18 and of an immunogenic composition comprising an antigen and a saponin adjuvant such as, but not limited to, QS-21 is extremely potent, and provides an efficient and well tolerated prophylaxis or treatment of infectious diseases, primary and metastatic neoplasic diseases (i.e. cancers), autoimmune diseases and related conditions, and is particularly effective in inhibiting the growth of human cancer cells that express a tumour-associated antigen.

### STATEMENT OF THE INVENTION

Accordingly there is provided a method for eliciting an enhanced immune response to an antigen in a patient, comprising administering to the patient a safe and effective amount of i) an immunogenic composition, in particular a vaccine, comprising an antigen or immunogenic derivative thereof and a saponin adjuvant, and ii) an IL-18 polypeptide or bioactive fragment or variant thereof. In another embodiment, the invention provides for a method for reducing the severity of a cancer in a patient, including treating pre-established tumours (primary tumours and metastatic tumours) or preventing from cancer recurrences, said method comprising administering to the patient in need thereof a safe and effective amount of i) an IL-18 polypeptide or bioactive fragment or variant thereof and ii) an immunogenic composition, in particular a vaccine, comprising a tumour associated antigen or immunogenic derivative thereof and a saponin adjuvant.

In one embodiment, the IL-18 polypeptide is a murine or a human IL-18 polypeptide or immunogenic fragment or variant thereof. In another embodiment the antigen is a tumour-associated antigen. In still another embodiment the saponin is a non-toxic fraction of Quil A, for example QS-17 or QS-21. In one example, the saponin is QS21. Accordingly, in one embodiment, the invention relates to a method for reducing the severity of a cancer in a patient, including treating pre-established tumours (primary tumours and metastatic tumours) or preventing from cancer recurrences, particularly carcinoma of the breast, lung (particularly non—small cell lung carcinoma), melanoma, colorectal, ovarian, prostate, bladder, head and neck squamous carcinoma, gastric and other GI (gastrointestinal), in particular oesophagus cancer, said method comprising administering to the mammal i) an immunogenic composition, in particular a vaccine, comprising a tumour-associated antigen or immunogenic derivative thereof and QS-21, and ii) IL-18 polypeptide or bioactive fragment or variant thereof.

The present invention also relates to a combined preparation comprising as active ingredients the following individual components: (1) an IL-18 polypeptide or bioactive fragment or variant thereof and (2) an immunogenic composition comprising an antigen and a saponin adjuvant, the active ingredients being for the simultaneous, separate or sequential use for the prophylaxis and/or treatment of infectious diseases, cancer, including primary tumours and metastatic tumours, autoimmune diseases and related conditions. In one embodiment the immunogenic composition within the combined preparation contains an additional immunostimulant chemical selected from the group comprising: cholesterol, 3D-MPL, an immunostimulatory oligonucleotide, aluminium hydroxide, aluminium phosphate, tocopherol, and an oil in water emulsion or a combination of two or more of the said adjuvants. For example, the additional adjuvant may be an immunostimulatory oligonucleotide.

In a related aspect the present invention also provides a pharmaceutical kit comprising as active ingredients the following individual components: (1) an IL-18 polypeptide or bioactive fragment or variant thereof and (2) an immunogenic composition comprising an antigen or immunogenic derivative thereof and a saponin adjuvant, the active ingredients being for the simultaneous, separate or sequential use for the prophylaxis and/or treatment of infectious diseases, cancer, including primary tumours and metastatic tumours, and auto-immune diseases.

The invention further relates to the use of (1) an IL-18 polypeptide or bioactive fragment or variant thereof and (2) an immunogenic composition comprising an antigen or immunogenic derivative thereof and a saponin adjuvant, in the manufacture of a medicament for achieving a protective immune response or reducing the severity of a disease in a patient, by administering to said patient a safe and effective amount both components.

The present invention further relates to processes for making such immunogenic compositions, to the use of such compositions for the prevention and/or the treatment of diseases, in particular cancer, and to the use of such compositions to inhibit the growth of tumours or cancerous cells in mammals, including humans.

### DETAILED DESCRIPTION

In one form of the present invention, the saponin adjuvant within the immunogenic composition is a non-toxic fraction of Quil A, for example QS-17 or QS-21, for example QS-21. The antigen may be an antigen derived from an infectious organism, for example a tumour-associated antigen or immunogenic derivative or derivative thereof. In one embodiment, the TH-1 cytokine is murine or human IL-18 or bioactive fragment thereof. The immunogenic composition and IL-may 18 act synergically in the induction of antigen-specific antibody, and may be potent in inducing or enhancing humoral or/and cellular immune responses conventionally associated with the TH-1 type immune system. By enhancement of immune response is meant the total increase in the immune response, as determined by humoral and/or cell mediated immune response, or by reduction of the tumour size and/or load. By synergy is meant that the IL-18 polypeptide or immunogenic fragment or variant thereof is capable of inducing an immune response when administered in a combined therapy with the immunogenic composition of the invention, and the presence of such immunogenic composition may enhance the efficacy of the IL-18 polypeptide or immunogenic fragment or variant thereof. The outcome of induction of the immune response may be prophylaxis, reduction of the severity of the disease (including, in the case of cancer, reduction of pre-established tumours, primary or metastasis, or prevention of cancer recurrences), and/or therapy.

Accordingly, in one embodiment, there is provided a method for eliciting an immune response to an antigen in a mammal, comprising administering to the mammal i) an effective amount of an immunogenic composition comprising an antigen derived from an infectious organism, or a tumour-associated antigen and QS-21, and ii) an IL-18 polypeptide or bioactive fragment or variant thereof. In one embodiment, both components of the treatment are given sequentially. That is said immunogenic composition is used to boost a humoral and/or a cellular immune response primed by the administration of IL-18. Alternatively, in another embodiment, the immunogenic composition according to the invention is used to prime a humoral and/or a cellular immune response in an individual who will subsequently receive IL-18. In still another embodiment both components of said treatment are given simultaneously, either through co-administration in two different sites or admixed within the same preparation. The skilled man will understand that both the immunogenic composition and the IL-18 polypeptide or immunogenic fragment or variant thereof may be given once or repetitively.

The combination therapy as contemplated within the scope of the present invention is at least as effective, for example of increased efficacy, compared to either component used alone. Especially in the field of cancer, the combined treatment is advantageous because it combines two anti-cancer agents, each operating in an additive fashion, for example synergistically, via a different mechanism of action to yield an enhanced cytotoxic response against human tumour cells.

In a related embodiment there is provided a combined preparation (for example a pharmaceutical multivial pack) comprising as active ingredients (1) an IL-18 polypeptide or bioactive fragment or variant thereof and (2) an immunogenic composition comprising an antigen and a saponin adjuvant, the active ingredients being for the simultaneous, separate or sequential use for the prophylaxis and/or treatment of infectious diseases, and cancer.

By combined preparation is meant a pharmaceutical preparation, or a pharmaceutical (multivial) pack or dispenser device which may contain one or more unit dosage forms containing the active ingredients. The pack may for example comprise metal or plastic foil, such as a blister pack. The pack or dispenser device may be accompanied by instructions for administration. Where the IL-18 polypeptide or bioactive fragment or variant thereof and the immunogenic composition are intended for administration as two separate compositions these may be presented in the form of, for example, a multivial pack. The active ingredients which are administered either at the same time, or separately, or sequentially, according to the invention, do not represent a mere aggregate of known agents, but a new combination with the surprising valuable property that the use of an IL-18 polypeptide or immunogenic fragment or variant thereof, allows the simulation of both the innate and adaptive components of the immune system, including NK cell activation as well as T cell mediated immune responses and cytokine production, thereby increasing the efficacy of the immunogenic composition. This results into a new and effective treatment. It is to be understood that the combined preparation, also designated as a kit-of-parts, means that the components of the combined preparation are not necessarily present as a union e.g. in composition, in order to be available for separate or sequential application. Thus the expression of kit-of-parts means that it is not necessarily a true combination, in view of the physical separation of the components.

The combined preparation may be used for either the treatment or prophylaxis of cancer, in particular for the reduction of the severity of cancer or the prevention of cancer recurrences. Cancers that can benefit from the combined therapy as herein described include any disease characterised by uncontrolled cell growth and proliferation, preneoplastic lesions, primary tumours and metastatic neoplastic lesions, and include, but are not limited to breast carcinoma, lung (particularly non small cell lung—NSCLC—carcinoma), melanoma, colorectal, ovarian, prostate, bladder, head and neck squamous carcinoma, gastric and other GI (gastrointestinal), in particular oesophagus cancer, leukemia, lymphoma, myeloma and plasmacytoma.

Exemplary antigens or derivative and fragments thereof, including peptides (i.e. less than about 50 amino acids), include the antigens encoded by the family of MAGE (Melanoma Antigen-encoding Genes) which are known as cancer (-testis) antigens (Gaugler B. et al. J. Exp. Med., 1994, 179: 921; Weynants P. et al. Int. J. Cancer, 1994, 56: 826; Patard J. J. et al. Int. J. Cancer, 1995, 64: 60). Cancers expressing MAGE proteins are known as MAGE-associated tumours. MAGE genes belong to a family of closely related genes, including i.e. MAGE 1, MAGE 2, MAGE 3 (Melanoma Antigen-encoding Gene-3), MAGE 4, MAGE 5, MAGE 6, MAGE 7, MAGE 8, MAGE 9, MAGE 10, MAGE 11, MAGE 12, located on chromosome X and sharing with each other 64 to 85% homology in their coding sequence (De Plaen E. et al., Immunogenetics, 1994, 40, 360-369). These are sometimes known as MAGE A1, MAGE A2, MAGE A3, MAGE A4, MAGE A5, MAGE A6, MAGE A7, MAGE A8, MAGE A9, MAGE A 10, MAGE A11, MAGE A 12 (The MAGE A family). Two other groups of proteins are also part of the MAGE family although more distantly related. These are the MAGE B and MAGE C group. The MAGE B family includes MAGE B1 (also known as MAGE Xp1, and DAM 10), MAGE B2 (also known as MAGE Xp2 and DAM 6) MAGE B3 and MAGE B4—the MAGE C family currently includes MAGE C1 and MAGE C2. In general terms, a MAGE protein can be defined as containing a core sequence signature located towards the C-terminal end of the protein (for example with respect to MAGE A1, a 309 amino acid protein, the core signature corresponds to amino acid 195-279).

The consensus pattern of the core signature is thus described as follows wherein x represents any amino acid, lower case residues are conserved (conservative variants allowed) and upper case residues are perfectly conserved.

Core Sequence Signature:
LixvL(2x)↑(3x)g(2x)apEExiWexl(2x)m(3-4x)Gxe(3-4x)gxp(2x)∥t(3x)VqexYLxYxqVPxsxP(2x)yeFLWGprA(2x)Et(3x)kv

Conservative substitutions are well known and are generally set up as the default scoring matrices in sequence alignment computer programs. These programs include PAM250 (Dayhoft M. O. et al., 1978, “A model of evolutionary changes in proteins”, In “Atlas of Protein sequence and structure” 5(3) M. O. Dayhoft (ed.), 345-352), National Biomedical Research Foundation, Washington, and Blosum 62 (Steven Henikoft & Jorja G. Henikoft (1992), “Amino acid substitution matricies from protein blocks”), Proc. Natl. Acad. Sci. USA 89 (Biochemistry): 10915-10919.

In general terms, substitution within the following groups are conservative substitutions, but substitutions between groups are considered non-conserved. The groups are:
i) Aspartate/asparagine/glutamate/glutamine
ii) Serine/threonine
iii) Lysine/arginine
iv) Phenylalanine/tyrosine/tryptophane
v) Leucine/isoleucine/valine/methionine
vi) Glycine/alanine

In general and in the context of this invention, a MAGE protein will be approximately 50% identical in this core region with amino acids 195 to 279 of MAGE A1.

MAGE-3 is expressed in 69% of melanomas (Gaugler B. et al. J. Exp. Med., 1994, 179: 921), and can also be detected in 44% of NSCLC (Yoshimatsu T. J Surg Oncol., 1998, 67,126-129), 75% of small cell lung cancers (SCLC) (Traversari C. et al., Gene Ther. 1997, 4: 1029-1035), 48% of head and neck squamous cell carcinoma, 34% of bladder transitional cell carcinoma 57% of oesophagus carcinoma 32% of colon cancers and 24% of breast cancers (Van Pel A. et al., Immunol. Rev., 1995, 145: 229; Inoue H. et al. Int. J. Cancer, 1995, 63: 523; Nishimura S et al., Nihon Rinsho Meneki Gakkai Kaishi Apr. 20, 1997 (2): 95-101). Several CTL epitopes have been identified on the MAGE-3 protein which have specific binding motifs for either the MHC class I molecule HLA.A1, or HLA.A2 (Van der Bruggen P. et al., Eur. J. Immunol., 1994, 24, 3038-3043) and HLA.B44 (Herman, J. et al., Immunogenetics, 1996, 43, 377-383) alleles respectively.

Although MAGE-3 has been detected in melanoma, lung and esophageal cancers, the level of expression of these antigens in patients with MAGE-associated tumours appears to be limited and below the threshold for immune recognition (Weiser T. S. et al., Ann. Thorac. Surg. 2001, 71: 295-302).

Other exemplary antigens or derivatives or fragments derived therefrom include MAGE antigens such as disclosed in WO 99/40188, PRAME (WO 96/10577), BAGE, RAGE, LAGE 1 (WO 98/32855), LAGE 2 (also known as NY-ESO-1, WO 98/14464), XAGE (Liu et al, Cancer Res, 2000, 60:4752-4755; WO 02/18584) SAGE, and HAGE (WO 99/53061) or GAGE (Robbins and Kawakami, 1996, Current Opinions in Immunology 8, pps 628-636; Van den Eynde et al., International Journal of Clinical & Laboratory Research (submitted 1997); Correale et al. (1997), Journal of the National Cancer Institute 89, p 293. Indeed these antigens are expressed in a wide range of tumour types such as melanoma, lung carcinoma, sarcoma and bladder carcinoma.

In one embodiment prostate antigens are utilised, such as prostate cancer antigens or prostate specific differentiation antigen (PSA), PAP, PSCA (PNAS 95(4) 1735-1740 1998), PSMA or the antigen known as prostase.

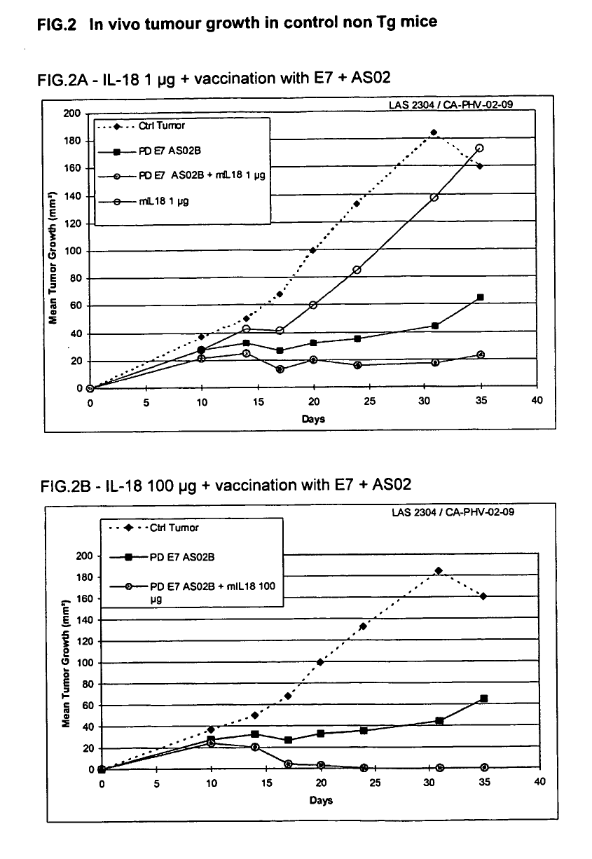

In another embodiment, the prostate antigen is P501S or a fragment thereof. P501S, also named prostein (Xu et al., Cancer Res. 61, 2001, 1563-1568), is known as SEQ ID NO. 113 of WO98/37814 and is a 553 amino acid protein. Immunogenic fragments and portions thereof comprising 20 or at least 20, 50 or at least 50, or 100 or at least 100 contiguous amino acids as disclosed in the above referenced patent application and are specifically contemplate by the present invention. Fragments which may be used are disclosed in WO 98/50567 (PS108 antigen) and as prostate cancer-associated protein (SEQ ID NO: 9 of WO 99/67384). Other fragments which may be used are amino acids 51-553, 34-553 or 55-553 of the full-length P501S protein. In particular, construct 1, 2 and 3 (see FIG. 2, SEQ ID NOs. 27-32) may be contemplated, and can be expressed in yeast systems, for example DNA sequences encoding such polypeptides can be expressed in yeast system.

Prostase is a prostate-specific serine protease (trypsin-like), 254 amino acid-long, with a conserved serine protease catalytic triad H-D-S and a amino-terminal pre-propeptide sequence, indicating a potential secretory function (P. Nelson, Lu Gan, C. Ferguson, P. Moss, R. Iinas, L. Hood & K. Wand, “Molecular cloning and characterisation of prostase, an androgen-regulated serine protease with prostate restricted expression, In Proc. Natl. Acad. Sci. USA (1999) 96, 3114-3119). A putative glycosylation site has been described. The predicted structure is very similar to other known serine proteases, showing that the mature polypeptide folds into a single domain. The mature protein is 224 amino acids-long, with at least one A2 epitope shown to be naturally processed. Prostase nucleotide sequence and deduced polypeptide sequence and homologous are disclosed in Ferguson, et al. (Proc. Natl. Acad. Sci. USA 1999, 96, 3114-3119) and in International Patent Applications No. WO 98/12302 (and also the corresponding granted patent U.S. Pat. No. 5,955,306), WO 98/20117 (and also the corresponding granted patents U.S. Pat. No. 5,840,871 and U.S. Pat. No. 5,786,148) (prostate-specific kallikrein) and WO 00/04149 (P703P).

Other prostate specific antigens are known from WO98/37418, and WO/004149. Another is STEAP (PNAS 96 14523 14528 7-12 1999).

Other tumour associated antigens useful in the context of the present invention include: Plu-1 J Biol. Chem 274 (22) 15633-15645, 1999, HASH-1, HASH-2 (Alders, M. et al., Hum. Mol. Genet. 1997, 6, 859-867), Cripto (Salomon et al Bioessays 199, 21 61-70,U.S. Pat. No. 5,654,140), CASB616 (WO 00/53216), Criptin (U.S. Pat. No. 5,981,215). Additionally, antigens particularly relevant for vaccines in the therapy of cancer also comprise tyrosinase, telomerase, P53, NY-Br1.1 (WO 01/47959) and fragments thereof such as disclosed in WO 00/43420, B726 (WO 00/60076, SEQ ID nos 469 and 463; WO 01/79286, SEQ ID nos 474 and 475), P510 (WO 01/34802 SEQ ID nos 537 and 538) and survivin.

The present invention is also useful in combination with breast cancer antigens such as Her-2/neu, mammaglobin (U.S. Pat. No. 5,668,267), B305D (WO00/61753 SEQ ID nos 299, 304, 305 and 315), or those disclosed in WO00/52165, WO99/33869, WO99/19479, WO 98/45328. Her-2/neu antigens are disclosed inter alia, in U.S. Pat. No. 5,801,005. The Her-2/neu may comprise the entire extracellular domain (comprising approximately amino acid 1-645) or fragments thereof and at least an immunogenic portion of or the entire intracellular domain approximately the C terminal 580 amino acids. In particular, the intracellular portion may comprise the phosphorylation domain or fragments thereof. Such constructs are disclosed in WO00/44899. One construct which may be used is known as ECD-PhD, a second is known as ECD deltaPhD (see WO00/44899) also named dHer2. The Her-2/neu as used herein may be derived from, for example, rat, mouse or human.

Certain tumour antigens are small peptide antigens (i.e. less than about 50 amino acids). Exemplary peptides included Mucin-derived peptides such as MUC-1 (see for example U.S. Pat. No. 5,744,144; U.S. Pat. No. 5,827,666; WO88/05054, U.S. Pat. No. 4,963,484). Specifically contemplated are MUC-1 derived peptides that comprise at least one repeat unit of the MUC-1 peptide, or at least two such repeats and which is recognised by the SM3 antibody (U.S. Pat. No. 6,054,438). Other mucin derived peptides include peptide from MUC-5.

Alternatively, said antigen may be an interleukin such as IL13 and IL14. Or said antigen maybe a self peptide hormone such as whole length Gonadotrophin hormone releasing hormone (GnRH, WO95/20600), a short 10 amino acid long peptide, useful in the treatment of many cancers, or in immunocastration. Other tumour-specific antigens include, but are not restricted to tumour-specific gangliosides such as GM2, and GM3.

The antigen may also be derived from sources which are pathogenic to humans, such as Human Immunodeficiency virus HIV-1 (such as tat, nef, reverse transcriptase, gag, gp120 and gp160), human herpes simplex viruses, such as gD or derivatives thereof or Immediate Early protein such as ICP27 from HSV1 or HSV2, cytomegalovirus ((esp Human)(such as gB or derivatives thereof), Rotavirus (including live-attenuated viruses), Epstein Barr virus (such as gp350 or derivatives thereof), Varicella Zoster Virus (such as gpl, II and IE63), or from a hepatitis virus such as hepatitis B virus (for example Hepatitis B Surface antigen or a derivative thereof), hepatitis A virus, hepatitis C virus and hepatitis E virus, or from other viral pathogens, such as paramyxoviruses: Respiratory Syncytial virus (such as F and G proteins or derivatives thereof, parainfluenza virus, measles virus, mumps virus, human papilloma viruses (for example HPV6, 11, 16, 18, ..), flaviviruses (e.g. Yellow Fever Virus, Dengue Virus, Tick-borne encephalitis virus, Japanese Encephalitis Virus) or Influenza virus (whole live or inactivated virus, split influenza virus, grown in eggs or MDCK cells, or whole flu virosomes (as described by R. Gluck, Vaccine, 1992, 10, 915-920) or purified or recombinant proteins thereof, such as HA, NP, NA, or M proteins, or combinations thereof, or derived from bacterial pathogens such as Neisseria spp, including N. gonorrhea and N. meningitidis (for example capsular polysaccharides and conjugates thereof, transferrin-binding proteins, lactoferrin binding proteins, PilC, adhesins); S. pyogenes (for example M proteins or fragments thereof, C5A protease, lipoteichoic acids), S. agalactiae, S. mutans; H. ducreyi; Moraxella spp, including M catarrhalis, also known as Branhamella catarrhalis (for example high and low molecular weight adhesins and invasins); Bordetella spp, including B. pertussis (for example pertactin, pertussis toxin or derivatives thereof, filamenteous hemagglutinin, adenylate cyclase, fimbriae), B. parapertussis and B. bronchiseptica; Mycobacterium spp., including M. tuberculosis (for example ESAT6, Antigen 85A, -B or -C), M. bovis, M. leprae, M. avium, M. paratuberculosis, M. smegmatis; Legionella spp, including L. pneumophila; Escherichia spp, including enterotoxic E. coli (for example colonization factors, heat-labile toxin or derivatives thereof, heat-stable toxin or derivatives thereof), enterohemorragic E. coli, enteropathogenic E. coli (for example shiga toxin-like toxin or derivatives thereof; Vibrio spp, including V. cholera (for example cholera toxin or derivatives thereof); Shigella spp, including S. sonnet, S. dysenteriae, S. flexnerii; Yersinia spp, including Y. enterocolitica (for example a Yop protein) , Y. pestis, Y. pseudotuberculosis; Campylobacter spp, including C. jejuni (for example toxins, adhesins and invasins) and C. coli; Salmonella spp, including S. typhi, S. paratyphi, S. choleraesuis, S. enteritidis; Listeria spp., including L. monocytogenes; Helicobacter spp, including H. pylori (for example urease, catalase, vacuolating toxin); Pseudomonas spp, including P. aeruginosa; Staphylococcus spp., including S. aureus, S. epidernidis; Enterococcus spp., including E. faecalis, E. faecium; Clostridium spp., including C. tetani (for example tetanus toxin and derivative thereof), C. botulinum (for example botulinum toxin and derivative thereof), C. difficile (for example clostridium toxins A or B and derivatives thereof; Bacillus spp., including B; anthracis (for example botulinum toxin and derivatives thereof); Corynebacterium spp., including C. diphtheriae (for example diphtheria toxin and derivatives thereof); Borrelia spp., including B. burgdorferi (for example OspA, OspC, DbpA, DbpB), B. garinii (for example OspA, OspC, DbpA, DbpB), B. afzelii (for example OspA, OspC, DbpA, DbpB), B. andersonii (for example OspA, OspC, DbpA, DbpB), B. hermsii; Ehrlichia spp., including E. equi and the agent of the Human Granulocytic Ehrlichiosis; Rickettsia spp, including R. rickettsii; Chiamydia spp., including C. trachomatis (for example MOMP, heparin-binding proteins), C. pneumoniae (for example MOMP, heparin-binding proteins), C. psittaci;. Leptospira spp., including L. interrogans; Treponema spp., including T. pallidum (for example the rare outer membrane proteins), T. denticola, T. hyodysenteriae; or derived from parasites such as Plasmodium spp., including P. falciparum; Toxoplasma spp., including T. gondii (for example SAG2, SAG3, Tg34); Entamoeba spp., including E. histolytica; Babesia spp., including B. microti; Trypanosoma spp., including T. cruzi; Giardia spp., including G. lambia; Leshmania spp., including L. major; Pneumocystis spp., including P. carinii; Trichomonas spp., including T. vaginalis; Schisostoma spp., including S. mansoni, or derived from yeast such as Candida spp., including C. albicans; Cryptococcus spp., including C. neoformans.

Other antigens for M. tuberculosis are for example Tb Ra12, Tb H9, Tb Ra35, Tb38-1, Erd 14, DPV, MTI, MSL, mTTC2 and hTCC1 (WO 99/51748). Proteins for M. tuberculosis also include fusion proteins and variants thereof where at least two, or at least three polypeptides of M. tuberculosis are fused into a larger protein. Examples of fusions include Ra12-TbH9-Ra35, Erd14-DPV-MTI, DPV-MTI-MSL, Erd14-DPV-MTI-MSL-mTCC2, Erd14-DPV-MTI-MSL, DPV-MTI-MSL-mTCC2, TbH9-DPV-MTI (WO 99/51748).

Antigens for Chiamydia include for example the High Molecular Weight Protein (HWMP) (WO 99/17741), ORF3 (EP 366 412), and putative membrane proteins (Pmps). Other Chlamydia antigens of the vaccine formulation can be selected from the group described in WO 99/28475.

Bacterial antigens may be derived from Streptococcus spp, including S. pneumoniae (for example capsular polysaccharides and conjugates thereof, PsaA, PspA, streptolysin, choline-binding proteins) and the protein antigen Pneumolysin (Biochem Biophys Acta, 1989, 67, 1007; Rubins et al., Microbial Pathogenesis, 25, 337-342), and mutant detoxified derivatives thereof (WO 90/06951; WO 99/03884). Other bacterial antigens are derived from Haemophilus spp., including H. influenzae type B (for example PRP and conjugates thereof), non typeable H. influenzae, for example OMP26, high molecular weight adhesins, P5, P6, protein D and lipoprotein D, and fimbrin and fimbrin derived peptides (U.S. Pat. No. 5,843,464) or multiple copy variants or fusion proteins thereof.

Derivatives of Hepatitis B Surface antigen are well known in the art and include, inter alia, those PreS1, PreS2 S antigens set forth described in European Patent applications EP-A-414 374; EP-A-0304 578, and EP 198474. In one embodiment the HBV antigen is HBV polymerase (Ji Hoon Jeong et al , 1996, BBRC 223, 264-271; Lee H. J. et al , Biotechnol. Lett. 15, 821-826). HIV-derived antigens are also contemplated, such as HIV-1 antigen gp120, especially when expressed in CHO cells.

The immunogenic composition of the invention may comprise an antigen derived from the Human Papilloma Virus (HPV 6a, 6b, 11, 16, 18, 31, 33, 35, 39, 45, 51, 52, 56, 58, 59 and 68), in particular those HPV serotypes considered to be responsible for genital warts (HPV 6 or HPV 11 and others), and the HPV viruses responsible for cervical cancer (HPV16, HPV18 and others). Suitable HPV antigens are E1, E2, E4, E5, E6, E7, L1 and L2. Examples of forms of genital wart prophylactic, or therapeutic, fusions comprise L1 particles or capsomers, and fusion proteins comprising one or more antigens selected from the HPV 6 and HPV 11 proteins E6, E7, L1, and L2. Examples of fusion proteins are: L2E7 as disclosed in WO96/26277, and proteinD(1/3)-E7 disclosed in WO99/10375. A HPV cervical infection or cancer, prophylaxis or therapeutic vaccine, composition may comprise HPV 16 or 18 antigens. For example, L1 or L2 antigen monomers, or L1 or L2 antigens presented together as a virus like particle (VLP) or the L1 alone protein presented alone in a VLP or caposmer structure. Such antigens, virus like particles and capsomer are per se known. See for example WO94/00152, WO94/20137, WO94/05792, and WO093/02184.

Additional early proteins may be included alone or as fusion proteins such as E7, E2 or E5 for example; other embodiments include a VLP comprising L1E7 fusion proteins (WO96/11272). HPV 16 antigens may comprise the early proteins E6 or E7 in fusion with a protein D carrier to form Protein D-E6 or E7 fusions from HPV 16, or combinations thereof; or combinations of E6 or E7 with L2 (WO96/26277). Alternatively the HPV 16 or 18 early proteins E6 and E7, may be presented in a single molecule, for example a Protein D-E6/E7 fusion. Other fusions optionally contain either or both E6 and E7 proteins from HPV 18, for example in the form of a Protein D-E6 or Protein D-E7 fusion protein or Protein D E6/E7 fusion protein. Fusions may comprise antigens from other HPV strains, for example from strains HPV 31 or 33.

Antigens derived from parasites that cause Malaria are also contemplated. For example, antigens from Plasmodia falciparum include RTS,S and TRAP. RTS is a hybrid protein comprising substantially all the C-terminal portion of the circumsporozoite (CS) protein of P. falciparum linked via four amino acids of the preS2 portion of Hepatitis B surface antigen to the surface (S) antigen of hepatitis B virus. Its full structure is disclosed in WO93/10152. When expressed in yeast RTS is produced as a lipoprotein particle, and when it is co-expressed with the S antigen from HBV it produces a mixed particle known as RTS,S. TRAP antigens are described in WO90/01496. An embodiment of the present invention is a fusion wherein the antigenic preparation comprises a combination of the RTS,S and TRAP antigens. Other plasmodia antigens that are likely candidates to be components of the fusion are P. faciparum MSP1, AMA1, MSP3, EBA, GLURP, RAP1, RAP2, Sequestrin, PfEMP1, Pf332, LSA1, LSA3, STARP, SALSA, PfEXP1, Pfs25, Pfs28, PFS27/25, Pfs16, Pfs48/45, Pfs230 and their analogues in Plasmodium spp.

As used herein, the term “immunogenic composition” is used in its broadest sense to mean an composition that, upon administration to a patient, positively affects the immune response of said patient. An immunogenic composition provides the patient with enhanced systemic or local immune response, either cellular immune responses such as CTL or humoral immune responses such as elicitation of antibodies. In particular, an immunogenic composition according to the present invention may refer to a formulation comprising an effective amount of an antigen polypeptide/protein, for example a tumour-associated antigen, and immunogenic derivative thereof, for example fragments thereof, or the encoding polynucleotide and a pharmaceutically acceptable carrier. By safe and effective amount is meant a dose of protein that, if necessary in association with an adjuvant, when administered to a human, produces a detectable immune response, such as a humoral response (antibodies) or a cellular response, or has the capacity to immunomodulate the immune system, without significant adverse side effects in typical vaccinees. Such amount will vary depending upon which specific immunogen is employed and how it is presented. Generally, it is expected that each dose will comprise 1-5000 μg of protein, for example 1-1000 μg of protein, for example 1-500 μg, for example 1-100 μg, for example 1 to 50 μg. An optimal amount for a particular vaccine can be ascertained by standard studies involving observation of appropriate immune responses in subjects. Following an initial vaccination, subjects may receive one or several booster immunisations adequately spaced. Vaccine preparation is generally described in Vaccine Design (“The subunit and adjuvant approach” (eds. Powell M. F. & Newman M. J). (1995) Plenum Press New York). Encapsulation within liposomes is described by Fullerton, U.S. Pat. No. 4,235,877.

Immunogenic antigen polypeptides refer to polypeptide which react detectably within an immunoassay (such as an ELISA or T-cell stimulation assay) with antisera and/or T-cells from a patient who expresses said polypeptide. Screening for immunogenic activity can be performed using techniques well known to the skilled artisan. For example, such screens can be performed using methods such as those described in Harlow and Lane, Antibodies: A Laboratory Manual, Cold Spring Harbor Laboratory, 1988. In one illustrative example, a polypeptide may be immobilised on a solid support and contacted with patient sera to allow binding of antibodies within the sera to the immobilised polypeptide. Unbound sera may then be removed and bound antibodies detected using, for example, ¹²⁵I-labeled Protein A.

The polypeptide antigens of the present invention are provided, for example, at least 80% pure for example 90% pure as visualised by SDS PAGE. The polypeptide antigens may appear as a single band by SDS PAGE.

Immunogenic derivatives such as immunogenic fragments or portions of antigens, for example of tumour associated or tumour specific antigen are also encompassed by the present invention. An “immunogenic fragment” as used herein, is a fragment that itself is immunologically reactive (i.e., specifically binds) with the B-cells and/or T-cell surface antigen receptors that recognize the polypeptide. Immunogenic portions may generally be identified using well known techniques, such as those summarized in Paul, Fundamental Immunology, 3rd ed., 243-247 (Raven Press, 1993) and references cited therein. Such techniques include screening polypeptides for the ability to react with antigen-specific antibodies, antisera and/or T-cell lines or clones.

As used herein, antisera and antibodies are “antigen-specific” if they specifically bind to an antigen (i.e., they react with the protein in an ELISA or other immunoassay, and do not react detectably with unrelated proteins). Such antisera and antibodies may be prepared as described herein, and using well-known techniques.

In one embodiment, an immunogenic portion of a polypeptide is a portion that reacts with antisera and/or T-cells at a level that is not substantially less than the reactivity of the full-length polypeptide (e.g., in an ELISA and/or T-cell reactivity assay). The level of immunogenic activity of the immunogenic portion may be at least about 50%, at least about 70%, or greater than about 90% of the immunogenicity for the full-length polypeptide. In some instances, immunogenic portions will be identified that have a level of immunogenic activity greater than that of the corresponding full-length polypeptide, e.g., having greater than about 100% or 150% or more immunogenic activity. In certain other embodiments, illustrative immunogenic portions may include peptides in which an N-terminal leader sequence and/or transmembrane domain have been deleted. Other illustrative immunogenic portions will contain a small N— and/or C-terminal deletion (e.g., about 1-50 amino acids, about 1-30 amino acids, about 5-15 amino acids), relative to the mature protein.

In another embodiment, illustrative immunogenic compositions, such as for example vaccine compositions, of the present invention comprise a polynucleotide encoding one or more of the polypeptides as described above, such that the polypeptide is generated in situ. The polynucleotide may be administered within any of a variety of known delivery systems. Indeed, numerous gene delivery techniques are well known in the art, such as those described by Rolland, Crit. Rev. Therap. Drug Carrier Systems 15:143-198, 1998, and references cited therein. Appropriate polynucleotide expression systems will, of course, contain the necessary regulatory DNA regulatory sequences for expression in a patient (such as a suitable promoter and terminating signal). Alternatively, bacterial delivery systems may involve the administration of a bacterium (such as Bacillus-Calmette-Guerrin) that expresses an immunogenic portion of the polypeptide on its cell surface or secretes such an epitope.

In one embodiment of the invention, a polynucleotide is administered/delivered as “naked” DNA, for example as described in Ulmer et al., Science 259:1745-1749, 1993 and reviewed by Cohen, Science 259:1691-1692, 1993. The uptake of naked DNA may be increased by coating the DNA onto biodegradable beads, which are efficiently transported into the cells. In one embodiment, the composition is delivered intradermally. In particular, the composition is delivered by means of a gene gun (particularly particle bombardment) administration techniques which involve coating the vector on to a bead (e.g. gold) which are then administered under high pressure into the epidermis; such as, for example, as described in Haynes et al, J Biotechnology 44: 37-42 (1996).

In one illustrative example, gas-driven particle acceleration can be achieved with devices such as those manufactured by Powderject Pharmaceuticals PLC (Oxford, UK) and Powderject Vaccines Inc. (Madison, Wis.), some examples of which are described in U.S. Pat. Nos. 5,846,796; 6,010,478; 5,865,796; 5,584,807; and EP Patent No. 0500 799. This approach offers a needle-free delivery approach wherein a dry powder formulation of microscopic particles, such as polynucleotide, are accelerated to high speed within a helium gas jet generated by a hand held device, propelling the particles into a target tissue of interest, typically the skin. The particles may be gold beads of a 0.4-4.0 μm, 0.6-2.0 μm diameter and the DNA conjugate coated onto these and then encased in a cartridge or cassette for placing into the “gene gun”.

In a related embodiment, other devices and methods that may be useful for gas-driven needle-less injection of compositions of the present invention include those provided by Bioject, Inc. (Portland, Oreg.), some examples of which are described in U.S. Pat. Nos. 4,790,824; 5,064,413; 5,312,335; 5,383,851; 5,399,163; 5,520,639 and 5,993,412.

Therefore, in certain embodiments, polynucleotides encoding immunogenic polypeptides described herein are introduced into suitable mammalian host cells for expression using any of a number of known viral-based systems. In one illustrative embodiment, retroviruses provide a convenient and effective platform for gene delivery systems. A selected nucleotide sequence encoding a polypeptide of the present invention can be inserted into a vector and packaged in retroviral particles using techniques known in the art. The recombinant virus can then be isolated and delivered to a subject. A number of illustrative retroviral systems have been described (e.g., U.S. Pat. No. 5,219,740; Miller and Rosman (1989) BioTechniques 7:980-990; Miller, A. D. (1990) Human Gene Therapy 1:5-14; Scarpa et al. (1991) Virology 180:849-852; Burns et al. (1993) Proc. Natl. Acad. Sci. USA 90:8033-8037; and Boris-Lawrie and Temin (1993) Cur. Opin. Genet. Develop. 3:102-109.

In addition, a number of illustrative adenovirus-based systems have also been described. Unlike retroviruses which integrate into the host genome, adenoviruses persist extrachromosomally thus minimizing the risks associated with insertional mutagenesis (Haj-Ahmad and Graham (1986) J. Virol. 57:267-274; Bett et al. (1993) J. Virol. 67:5911-5921; Mittereder et al. (1994) Human Gene Therapy 5:717-729; Seth et al. (1994) J. Virol. 68:933-940; Barr et al. (1994) Gene Therapy 1:51-58; Berkner, K. L. (1988) BioTechniques 6:616-629; and Rich et al. (1993) Human Gene Therapy 4:461-476). Since humans are sometimes infected by common human adenovirus serotypes such as AdHu5, a significant proportion of the population have a neutralizing antibody response to the adenovirus, which is likley to effect the immune response to a heterologous antigen in a recombinant vaccine based system. Non-human primate adenoviral vectors such as the chimpanzee adenovirus 68 (AdC68, Fitzgerald et al. (2003) J. Immunol 170(3):1416-22)) are may offer an alternative adenoviral system without the disadvantage of a pre-existing neutralising antibody response.

Various adeno-associated virus (MV) vector systems have also been developed for polynucleotide delivery. MV vectors can be readily constructed using techniques well known in the art. See, e.g., U.S. Pat. Nos. 5,173,414 and 5,139,941; International Publication Nos. WO 92/01070 and WO 93/03769; Lebkowski et al. (1988) Molec. Cell. Biol. 8:3988-3996; Vincent et al. (1990) Vaccines 90 (Cold Spring Harbor Laboratory Press); Carter, B. J. (1992) Current Opinion in Biotechnology 3:533-539; Muzyczka, N. (1992) Current Topics in Microbiol. and Immunol. 158:97-129; Kotin, R. M. (1994) Human Gene Therapy 5:793-801; Shelling and Smith (1994) Gene Therapy 1:165-169; and Zhou et al. (1994) J. Exp. Med. 179:1867-1875.

Additional viral vectors useful for delivering the nucleic acid molecules encoding polypeptides of the present invention by gene transfer include those derived from the pox family of viruses, such as vaccinia virus and avian poxvirus. By way of example, vaccinia virus recombinants expressing the novel molecules can be constructed as follows. The DNA encoding a polypeptide is first inserted into an appropriate vector so that it is adjacent to a vaccinia promoter and flanking vaccinia DNA sequences, such as the sequence encoding thymidine kinase (TK). This vector is then used to transfect cells which are simultaneously infected with vaccinia. Homologous recombination serves to insert the vaccinia promoter plus the gene encoding the polypeptide of interest into the viral genome. The resulting TK.sup.(−) recombinant can be selected by culturing the cells in the presence of 5-bromodeoxyuridine and picking viral plaques resistant thereto.

A vaccinia-based infection/transfection system can be conveniently used to provide for inducible, transient expression or coexpression of one or more polypeptides described herein in host cells of an organism. In this particular system, cells are first infected in vitro with a vaccinia virus recombinant that encodes the bacteriophage T7 RNA polymerase. This polymerase displays exquisite specificity in that it only transcribes templates bearing T7 promoters. Following infection, cells are transfected with the polynucleotide or polynucleotides of interest, driven by a T7 promoter. The polymerase expressed in the cytoplasm from the vaccinia virus recombinant transcribes the transfected DNA into RNA which is then translated into polypeptide by the host translational machinery. The method provides for high level, transient, cytoplasmic production of large quantities of RNA and its translation products. See, e.g., Elroy-Stein and Moss, Proc. Natl. Acad. Sci. USA (1990) 87:6743-6747; Fuerst et al. Proc. Natl. Acad. Sci. USA (1986) 83:8122-8126.

Alternatively, avipoxviruses, such as the fowlpox and canarypox viruses, can also be used to deliver the coding sequences of interest. Recombinant avipox viruses, expressing immunogens from mammalian pathogens, are known to confer protective immunity when administered to non-avian species. The use of an Avipox vector is particularly desirable in human and other mammalian species since members of the Avipox genus can only productively replicate in susceptible avian species and therefore are not infective in mammalian cells. Methods for producing recombinant Avipoxviruses are known in the art and employ genetic recombination, as described above with respect to the production of vaccinia viruses. See, e.g., WO 91/12882; WO 89/03429; and WO 92/03545.

Any of a number of alphavirus vectors can also be used for delivery of polynucleotide compositions of the present invention, such as those vectors described in U.S. Pat. Nos. 5,843,723; 6,015,686; 6,008,035 and 6,015,694. Certain vectors based on Venezuelan Equine Encephalitis (VEE) can also be used, illustrative examples of which can be found in U.S. Pat. Nos. 5,505,947 and 5,643,576.

The vectors which comprise the nucleotide sequences encoding antigenic peptides are administered in such amount as will be prophylactically or therapeutically effective. The quantity to be administered maybe in the range of one picogram to 16 milligram, 1 picogram to 10 micrograms for particle-mediated delivery, and 10 micrograms to 16 milligram for other routes of nucleotide per dose. The exact quantity may vary considerably depending on the weight of the patient being immunised and the route of administration.

Suitable techniques for introducing the naked polynucleotide or vector into a patient also include topical application with an appropriate vehicle. The nucleic acid may be administered topically to the skin, or to mucosal surfaces for example by intranasal, oral, intravaginal or intrarectal administration. The naked polynucleotide or vector may be present together with a pharmaceutically acceptable excipient, such as phosphate buffered saline (PBS). DNA uptake may be further facilitated by use of facilitating agents such as bupivacaine, either separately or included in the DNA formulation. Other methods of administering the nucleic acid directly to a recipient include ultrasound, electrical stimulation, electroporation and microseeding which is described in U.S. Pat. No. 5,697,901.

Uptake of nucleic acid constructs may be enhanced by several known transfection techniques, for example those including the use of transfection agents. Examples of these agents include cationic agents, for example, calcium phosphate and DEAE-Dextran and lipofectants, for example, lipofectam and transfectam. The dosage of the nucleic acid to be administered can be altered.

In still another embodiment, the immunogenic compositions of the present invention comprise an antibody, or a serum, or a domain of an antibody such as Fab and F(ab′)2 fragment. The antibody may be a monoclonal antibody or fragment thereof. The effective dosage is typically 100 μg to 500 mg, for example 1 mg to 50 mg per kilo of patient body weight. Accordingly, the methods of the present invention include passive immunotherapy or passive immunoprophylaxis.

The immunogenic compositions and the IL-18 polypeptide of the present invention can be delivered by a number of routes such as intramuscularly, subcutaneously, intraperitonally or intravenously.

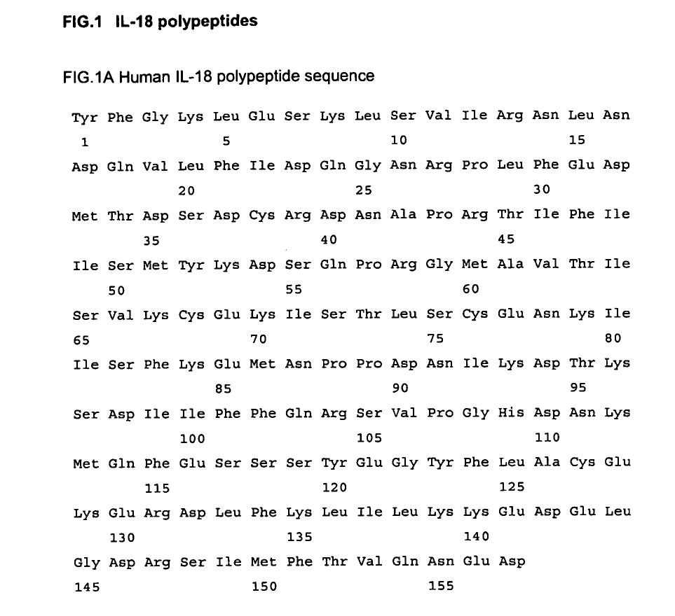

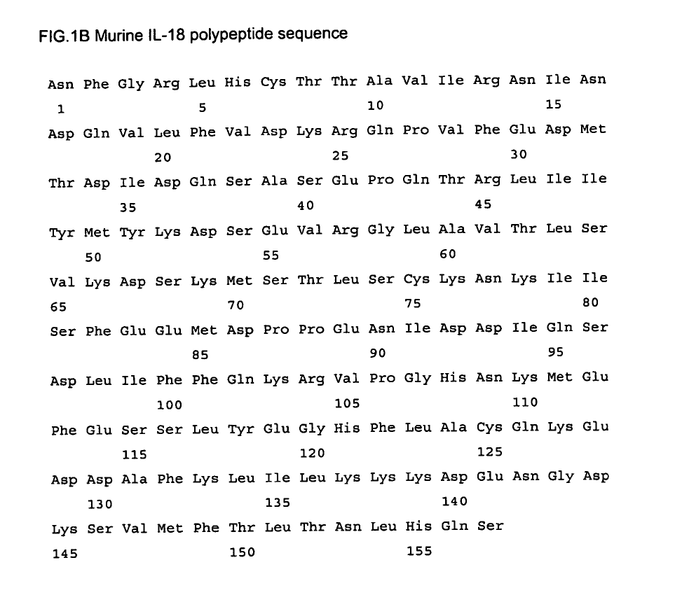

The IL-18 polypeptide or bioactive fragment thereof according to the present invention is one that induces an immune response predominantly of the Th1 type. High levels of Th1-type cytokines (e.g., IFN-γ, TNFα, IL-2, IL-12, IL-18, etc) tend to favor the induction of cell mediated immune responses to an administered antigen. In contrast, high levels of Th2-type cytokines (e.g., IL-4, IL-5, IL-6 and IL-10) tend to favor the induction of humoral immune responses. For a review of the families of cytokines, see Mosmann and Coffman, Ann. Rev. Immunol. 7:145-173, 1989. By “IL-18” or “IL-18 polypeptide” is meant a IL-18 polypeptide as disclosed in EP0692536, EP 0712931, EP0767178 and WO97/2441. IL-18 polypeptides derivatives or variants include isolated polypeptides comprising an amino acid sequence which has at least 70% identity, at least 80% identity, at least 90% identity, at least 95% identity, at least 97-99% identity to that of SEQ ID NO:6 (human IL-18) or SEQ ID NO:7 (murine IL-18) as depicted in FIG. 1, over the entire length of SEQ ID NO:6 and SEQ ID NO:7, respectively. Such polypeptides include those comprising the amino acid of SEQ ID NO:6 and SEQ ID NO:7, respectively. IL-18 polypeptide may be the amino acid sequence as set forth in SEQ ID NO:6 and SEQ ID NO:7. IL-18 fragments are also contemplated, that is a fragment of IL-18 which are capable of exhibiting a biological (antigenic or immunogenic) activity of IL-18 such as the induction of IFN-γ. IL-18 bioactive fragments may be used, IL-18 immunogenic fragments may be used.

IL-18 polypeptide may be in the form of mature protein or may be a part of larger protein such as a fusion protein. IL-18 variants are also contemplated, that is polypeptides that vary by conservative amino acid substitutions, whereby a residue is substituted by another with like characteristics. Typical such substitutions are among Ala, Val, Leu and Ile; among Ser and Thr; among the acidic residues Asp and Glu; among Asn and Gln; among the basic residues Lys and Arg; or aromatic residues Phe and Tyr. For example variants in which several, 5-10, 1-5, 1-3, 1-2 or 1 amino acids are substituted, deleted or added in any combination. IL-18 bioactive fragments are also contemplated. By “bioactive fragment” is meant a fragment of IL-18 which has retained substantially the same bioactivity as the full-length IL-18. By bioactivity is meant any of the following properties: augmentation of natural killer (NK) cell activity and Th1 cell response (activation of NK; NKT cells, induction of the proliferation of activated T cells), anti-angiogenic activity, enhancement of the expression of Fas ligand on activated NK, NKT cells and T cells, increased production of IFNg, GM-CSF and other cytokines for example of Th1-type, capacity to stimulate innate immunity and both Th1- and Th2-mediated responses.

In particular, a bioactive fragment of IL-18 is a fragment which has retained the ability to increase the production of IFNg as measured, in vitro, by KG-1 assay system. Human myelomonocytic cell line (KG-1), that express human IL-18 receptor, will respond to treatment with IL-18 by increasing the production (secretion) of IFNg (measured by ELISA) and activation of NfKB (Matsuko Taniguchi et al. J. Immunological Methods, 1998, 217, 97-102).

IL-18 polypeptides according to the present invention can be prepared in any suitable manner. The include isolating naturally occurring polypeptides, recombinantly or synthetically producing said polypeptides, etc. Such preparation means are well understood in the art.

The immunogenic compositions according to the invention may advantageously include a pharmaceutically acceptable excipient or carrier. A carrier molecule may encompass several forms, including a carrier organism such as a live bacterial vector or a bacterial carrier strain, water, saline or an immunostimulant chemical. A carrier can be water, saline or other buffered physiological solutions. A carrier molecule may also include a porous polymeric particle, such as a microbead or a nanoparticle, and a metallic salt particle such as aluminium hydroxide, aluminium phosphate or calcium phosphate, or magnesium phosphate, iron phosphate, calcium carbonate, magnesium carbonate, calcium sulfate, magnesium hydroxyde, or double salts like ammonium-iron phosphate, potassium-iron phosphate, calcium-iron phosphate, calcium-magnesium carbonate, or a mix of any of those salts.

Upon administration of the combined preparation as provided herein, a patient will support an immune response that includes Th1- and Th2-type responses.

The immunogenic compositions and the IL-18 polypeptide or bioactive fragment or variant thereof according to the present invention can be delivered by a number of routes such as intramuscularly, subcutaneously, intraperitonally or intravenously.

The vectors which comprise the nucleotide sequences encoding antigenic peptides are administered in such amount as will be prophylactically or therapeutically effective. The quantity to be administered, is generally in the range of one picogram to 16 milligram, for example 1 picogram to 10 micrograms for particle-mediated delivery, and 10 micrograms to 16 milligram for other routes of nucleotide per dose. The exact quantity may vary considerably depending on the weight of the patient being immunised and the route of administration.

Suitable techniques for introducing the naked polynucleotide or vector into a patient also include topical application with an appropriate vehicle. The nucleic acid may be administered topically to the skin, or to mucosal surfaces for example by intranasal, oral, intravaginal or intrarectal administration. The naked polynucleotide or vector may be present together with a pharmaceutically acceptable excipient, such as phosphate buffered saline (PBS). DNA uptake may be further facilitated by use of facilitating agents such as bupivacaine, either separately or included in the DNA formulation. Other methods of administering the nucleic acid directly to a recipient include ultrasound, electrical stimulation, electroporation and microseeding which is described in U.S. Pat. No. 5,697,901.

Uptake of nucleic acid constructs may be enhanced by several known transfection techniques, for example those including the use of transfection agents. Examples of these agents includes cationic agents, for example, calcium phosphate and DEAE-Dextran and lipofectants, for example, lipofectam and transfectam. The dosage of the nucleic acid to be administered can be altered.

The IL-18 polypeptide or bioactive fragment thereof according to the present invention is one that induces an immune response predominantly of the Th1 type. High levels of Th1-type cytokines (e.g., IFN-γ, TNFα, IL-2, IL-12, IL-18, etc) tend to favor the induction of cell mediated immune responses to an administered antigen. In contrast, high levels of Th2-type cytokines (e.g., IL4, IL-5, IL-6 and IL-10) tend to favor the induction of humoral immune responses. For a review of the families of cytokines, see Mosmann and Coffman, Ann. Rev. Immunol. 7:145-173, 1989. The levels of these cytokines may be readily assessed using standard assays. By “IL-18” is meant a IL-18 polypeptide as disclosed in EP0692536, EP 0712931, EP0767178 and WO97/2441. IL-18 polypeptides include isolated polypeptides comprising an amino acid sequence which has at least 70% identity, at least 80% identity, at least 90% identity, at least 95% identity, or at least 97-99% identity to that of SEQ ID NO:1 (human IL-18) or SEQ ID NO:2 (murine IL-18) as depicted in FIG. 1, over the entire length of SEQ ID NO:1 and SEQ ID NO:2, respectively. Such polypeptides include those comprising the amino acid of SEQ ID NO:1 and SEQ ID NO:2, respectively. IL-18 polypeptide may have the amino acid sequence as set forth in SEQ ID NO:1 and SEQ ID NO:2. IL-18 fragments are also contemplated, that is a fragment of IL-18 which are capable of exhibiting a biological (antigenic or immunogenic) activity of IL-18 such as the induction of IFN-γ.

IL-18 polypeptide may be in the form of mature protein or may be a part of larger protein such as a fusion protein. IL-18 variants are also contemplated, that is polypeptides that vary by conservative amino acid substitutions, whereby a residue is substituted by another with like characteristics. Typical such substitutions are among Ala, Val, Leu and Ile; among Ser and Thr; among the acidic residues Asp and Glu; among Asn and Gln; among the basic residues Lys and Arg; or aromatic residues Phe and Tyr. For example variants in which several, 5-10, 1-5, 1-3, 1-2 or 1 amino acids are substituted, deleted or added in any combination.

IL-18 polypeptides according to the present invention can be prepared in any suitable manner. The include isolating naturally occurring polypeptides, recombinantly or synthetically producing said polypeptides, etc. Such preparation means are well understood in the art.

Upon administration of the combined preparation as provided herein, a patient will support an immune response that includes Th1- and Th2-type responses.

Within embodiments of the invention, the immunogenic composition additionally comprises another adjuvant, for example one that induces an immune response predominantly of the Th1 type. TH-1 inducing adjuvants may be selected from the group of adjuvants comprising: lipopolysaccharide derived adjuvant such as enterobacterial lipopolysaccharide (LPS), 3D-MPL, cholesterol, and a CpG oligonucleotide or a mixture of two or more said adjuvants. Adjuvants for eliciting a predominantly Th1-type response may include, for example, a combination of monophosphoryl lipid A, for example 3-de-O-acylated monophosphoryl lipid A, together with an aluminum salt. MPL® adjuvants are available from Corixa Corporation (Seattle, Wash.; see, for example, U.S. Pat. Nos. 4,436,727; 4,877,611; 4,866,034 and 4,912,094).

In one embodiment, the immunogenic composition according to the invention additionally comprises an immunostimulatory CpG oligonucleotide.

CpG-containing oligonucleotides (in which the CpG dinucleotide is unmethylated) also induce a predominantly Th1response. Such oligonucleotides are well known and are described, for example, in WO 96/02555, WO 99/33488 and U.S. Pat. Nos. 6,008,200 and 5,856,462. Immunostimulatory DNA sequences are also described, for example, by Sato et al., Science 273:352,1996.

CpG's are known in the art as being adjuvants when administered by both systemic and mucosal routes (WO96/02555, EP 468520, Davis et al., J. Immunol, 1998, 160(2):870-876; McCluskie and Davis, J. Immunol., 1998, 161(9):4463-6). CpG is an abbreviation for cytosine-guanosine dinucleotide motifs present in DNA. Historically, it was observed that the DNA fraction of BCG could exert an anti-tumour effect. In further studies, synthetic oligonucleotides derived from BCG gene sequences were shown to be capable of inducing immunostimulatory effects (both in vitro and in vivo). The authors of these studies concluded that certain palindromic sequences, including a central CG motif, carried this activity. The central role of the CG motif in immunostimulation was later elucidated in a publication by Krieg, Nature 374, p546, 1995. Detailed analysis has shown that the CG motif has to be in a certain sequence context, and that such sequences are common in bacterial DNA but are rare in vertebrate DNA. The immunostimulatory sequence is often: Purine, Purine, C, G, pyrimidine, pyrimidine; wherein the dinucleotide CG motif is not methylated, but other unmethylated CpG sequences are known to be immunostimulatory and may be used in the present invention.

In certain combinations of the six nucleotides a palindromic sequence is present. Several of these motifs, either as repeats of one motif or a combination of different motifs, can be present in the same oligonucleotide. The presence of one or more of these immunostimulatory sequence containing oligonucleotides can activate various immune subsets, including natural killer cells (which produce interferon γ and have cytolytic activity) and macrophages (Wooldrige et al Vol 89 (no. 8), 1977). Although other unmethylated CpG containing sequences not having this consensus sequence have now been shown to be immunomodulatory.

The oligonucleotides for use in according to the present invention may contain two or more dinucleotide CpG motifs separated by at least three, at least six or more nucleotides. The oligonucleotides of the present invention may be deoxynucleotides. In one embodiment the internucleotide in the oligonucleotide is phosphorodithioate, or a phosphorothioate bond, although phosphodiester and other internucleotide bonds are within the scope of the invention including oligonucleotides with mixed internucleotide linkages. Methods for producing phosphorothioate oligonucleotides or phosphorodithioate are described in U.S. Pat. No. 5,666,153, U.S. Pat. No. 5,278,302 and WO95/26204.

Examples of oligonucleotides have the following sequences. The sequences may contain phosphorothioate modified internucleotide linkages.

OLIGO 1 (SEQ ID NO:3):
TCC ATG ACG TTC CTG ACG TT (CpG 1826)
OLIGO 2 (SEQ ID NO:4):
TCT CCC AGC GTG CGC CAT (CpG 1758)
OLIGO 3 (SEQ ID NO:5):
ACC GAT GAC GTC GCC GGT GAC GGC ACC ACG
OLIGO 4 (SEQ ID NO:6):
TCG TCG TTT TGT CGT TTT GTC GTT (CpG 2006 also
known as CpG 7909)
OLIGO 5 (SEQ ID NO:7):
TCC ATG ACG TTC CTG ATG CT (CpG 1668)

Alternative CpG oligonucleotides may comprise the sequences above in that they have inconsequential deletions or additions thereto.

The CpG oligonucleotides utilised in the present invention may be synthesized by any method known in the art (e.g. EP 468520). Conveniently, such oligonucleotides may be synthesized utilising an automated synthesizer.

The oligonucleotides utilised in the present invention may be deoxynucleotides. In one embodiment the internucleotide bond in the oligonucleotide is phosphorodithioate, or phosphorothioate bond, although phosphodiesters are within the scope of the present invention. Oligonucleotide comprising different internucleotide linkages are contemplated, e.g. mixed phosphorothioate phophodiesters. Other internucleotide bonds which stabilise the oligonucleotide may be used.

CpG when formulated into vaccines, is generally administered in free solution together with free antigen (WO 96/02555; McCluskie and Davis, supra) or covalently conjugated to an antigen (PCT Publication No. WO 98/16247), or formulated with a carrier such as aluminium hydroxide ((Hepatitis surface antigen) Davis et al. supra Brazolot-Millan et al., Proc. Natl. Acad. Sci., USA, 1998, 95(26), 15553-8).

One enhanced formulation may involve the combination of a CpG-containing oligonucleotide with a saponin derivative, particularly the combination of CpG and QS21 as disclosed in WO00/09159 and in WO00/62800. Such a formulation may additionally comprise an oil in water emulsion and tocopherol. Accordingly, in a yet further embodiment the immunogenic composition of the present invention comprises a combination of a CpG oligonucleotide and a saponin, for example QS21, optionally formulated in an oil in water emulsion. The formulation may optionally additionally comprise 3D-MPL® adjuvant. QS-21 may be provided in its less reactogenic composition where it is quenched with cholesterol, as described in WO 96/33739.

In another embodiment, the immunogenic composition of the present invention additionally comprise an enterobacterial lipopolysaccharide derived adjuvant, for example monophosphoryl lipid A, for example 3-de-O-acylated monophosphoryl lipid A.

It has long been known that enterobacterial lipopolysaccharide (LPS) is a potent stimulator of the immune system, although its use in adjuvants has been curtailed by its toxic effects. A non-toxic derivative of LPS, monophosphoryl lipid A (MPL), produced by removal of the core carbohydrate group and the phosphate from the reducing-end glucosamine, has been described by Ribi et al (1986, Immunology and Immunopharmacology of bacterial endotoxins, Plenum Publ. Corp., NY, p 407-419) and has the following structure:

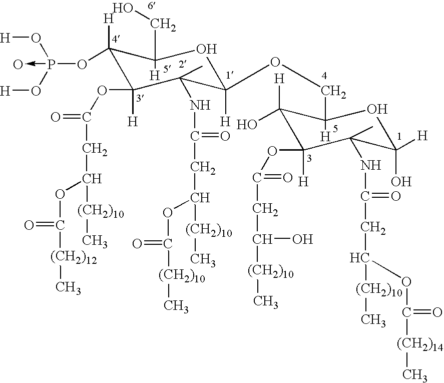

A further detoxified version of MPL results from the removal of the acyl chain from the 3-position of the disaccharide backbone, and is called 3-O-Deacylated monophosphoryl lipid A (3D-MPL). It can be purified and prepared by the methods taught in GB 2122204B, which reference also discloses the preparation of diphosphoryl lipid A, and 3-O-deacylated variants thereof. 3D-MPL may be in the form of an emulsion having a small particle size less than 0.2 μm in diameter, and its method of manufacture is disclosed in WO94/21292. Aqueous formulations comprising monophosphoryl lipid A and a surfactant have been described in WO98/43670A2.

The bacterial lipopolysaccharide derived adjuvants to be formulated in the adjuvant combinations of the present invention may be purified and processed from bacterial sources, or alternatively they may be synthetic. For example, purified monophosphoryl lipid A is described in Ribi et al 1986 (supra), and 3-O-Deacylated monophosphoryl or diphosphoryl lipid A derived from Salmonella sp. is described in GB 2220211 and U.S. Pat. No. 4,912,094. Other purified and synthetic lipopolysaccharides have been described (WO 98/01139; U.S. Pat. No. 6,005,099 and EP 0 729 473 B1; Hilgers et al., 1986, Int. Arch. Allergy. Immunol., 79(4):392-6; Hilgers et al., 1987, Immunology, 60(1):141-6; and EP 0 549 074 B1). Bacterial lipopolysaccharide adjuvants may include 3D-MPL and the β(1-6) glucosamine disaccharides described in U.S. Pat. No. 6,005,099 and EP 0 729 473 B1.

Accordingly, the LPS derivatives that may be used in the present invention are those immunostimulants that are similar in structure to that of LPS or MPL or 3D-MPL. In another aspect of the present invention the LPS derivatives may be an acylated monosaccharide, which is a sub-portion to the above structure of MPL.

An example of a disaccharide adjuvant is a purified or synthetic lipid A of the following formula: 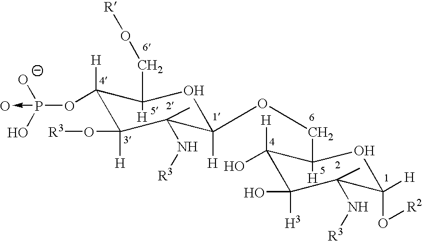
wherein R2 may be H or PO3H2; R3 may be an acyl chain or β-hydroxymyristoyl or a 3-acyloxyacyl residue having the formula: 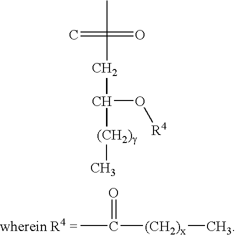
and wherein X and Y have a value of from 0 up to about 20.

Combinations of 3D-MPL and saponin adjuvants derived from the bark of Quillaja Saponaria molina have been described in EP 0 761 231 B. WO 95/17210 discloses an adjuvant emulsion system based on squalene, α-tocopherol, and polyoxyethylene sorbitan monooleate (TWEEN80), formulated with the immunostimulant QS21, optionally with 3D-MPL.

Accordingly, in another embodiment, the immunogenic composition according to the invention comprises (1) an antigen or immunogenic fragment thereof and (2) a combination of a saponin, for example QS21, for example in its quenched form with cholesterol, with one or more of the following adjuvants selected from the list comprising for example: a CpG immunostimulatory oligonucleotide, 3D-MPL, and an oil-in-water emulsion.

In one embodiment, the immunogenic composition includes the combination of a monophosphoryl lipid A to the saponin adjuvant, such as the combination of 3D-MPL® adjuvant with QS21, as described in WO94/00153, or a less reactogenic composition where the QS21 is quenched with cholesterol, as described in WO96/33739. Other formulations may comprise an oil-in-water emulsion and tocopherol in addition to QS-21. Another formulation employing a combination of QS21, 3D-MPL® adjuvant and tocopherol in an oil-in-water emulsion is described in WO95/17210. Accordingly the immunogenic composition according to the present invention comprises an antigen, for example a tumour-associated antigen, a saponin adjuvant, for example QS-21, together with 3D-MPL® adjuvant, optionally comprising an oil-in-water emulsion and tocopherol in addition to QS-21. For example QS-21 is quenched with cholesterol.

Another formulation involves the combination of a CpG-containing oligonucleotide with the saponin derivative, for example the combination of CpG and QS21 as disclosed in WO00/09159 and in WO00/62800. Such a formulation may additionally comprise an oil in water emulsion and tocopherol. Accordingly, in a yet further embodiment the immunogenic composition of the present invention comprises a combination of a saponin, for example QS21, and a CpG oligonucleotide, optionally formulated in an oil in water emulsion. The formulation may optionally additionally comprise 3D-MPL® adjuvant. QS-21 may be provided in its less reactogenic composition where it is quenched with cholesterol, as described in WO96/33739.

Alternatively the saponin adjuvant within the immunogenic composition according to the invention may be combined with vaccine vehicles composed of chitosan or other polycationic polymers, polylactide and polylactide-co-glycolide particles, poly-N-acetyl glucosamine-based polymer matrix, particles composed of polysaccharides or chemically modified polysaccharides, liposomes and lipid-based particles, particles composed of glycerol monoesters, etc. The saponins may also be formulated in the presence of cholesterol to form particulate structures such as liposomes or ISCOMs. Furthermore, the saponins may be formulated together with a polyoxyethylene ether or ester, in either a non-particulate solution or suspension, or in a particulate structure such as a paucilamelar liposome or ISCOM. The saponins may also be formulated with excipients such as CarbopoI^{R} to increase viscosity, or may be formulated in a dry powder form with a powder excipient such as lactose.

Vaccines and immunogenic compositions may be presented in unit-dose or multi-dose containers, such as sealed ampoules or vials. Such containers may be hermetically sealed to preserve sterility of the formulation until use. In general, formulations may be stored as suspensions, solutions or emulsions in oily or aqueous vehicles. Alternatively, a vaccine or immunogenic composition may be stored in a freeze-dried condition requiring only the addition of a sterile liquid carrier immediately prior to use.

Any of a variety of delivery vehicles may be employed within immunogenic compositions and vaccines to facilitate production of an antigen-specific immune response that targets tumour cells. According to one embodiment of this invention, the immunogenic composition described herein is delivered to a host via antigen-presenting cells (APCs), such as dendritic cells, macrophages, B cells, monocytes and other cells that may be engineered to be efficient APCs. APCs cells may, but need not, be genetically modified to increase the capacity for presenting the antigen, to improve activation and/or maintenance of the T cell response, to have anti-tumour effects per se and/or to be immunologically compatible with the receiver (i.e., matched HLA haplotype). APCs may generally be isolated from any of a variety of biological fluids and organs, including tumour and peri-tumoural tissues, and may be autologous, allogeneic, syngeneic or xenogeneic cells.

Certain embodiments of the present invention use dendritic cells or progenitors thereof as antigen-presenting cells. Dendritic cells are highly potent APCs (Banchereau J. & Steinman R. M., Nature, 1998, 392:245-251) and have been shown to be effective as a physiological adjuvant for eliciting prophylactic or therapeutic antitumour immunity (see Timmerman J. M. and Levy R., Ann. Rev. Med, 1999, 50:507-529). In general, dendritic cells may be identified based on their typical shape (stellate in situ, with marked cytoplasmic processes (dendrites) visible in vitro), their ability to take up, process and present antigens with high efficiency and their ability to activate naïve T cell responses. Dendritic cells may, of course, be engineered to express specific cell-surface receptors or ligands that are not commonly found on dendritic cells in vivo or ex vivo, and such modified dendritic cells are contemplated by the present invention. As an alternative to dendritic cells, secreted vesicles antigen-loaded dendritic cells (called exosomes) may be used within a vaccine (see Zitvogel L. et al., Nature Med., 1998, 4:594-600). Accordingly there is provided an immunostimulant formulation, for example a vaccine, comprising an effective amount of dendritic cells or antigen presenting cells, modified by in vitro loading with a polypeptide as described herein, or genetically modified in vitro to express a polypeptide as described herein and a pharmaceutically effective carrier.

Dendritic cells and progenitors may be obtained from peripheral blood, bone marrow, tumour-infiltrating cells, peritumoural tissues-infiltrating cells, lymph nodes, spleen, skin, umbilical cord blood or any other suitable tissue or fluid. For example, dendritic cells may be differentiated ex vivo by adding a combination of cytokines such as GM-CSF, IL4, IL-13 and/or TNFα to cultures of monocytes harvested from peripheral blood. Alternatively, CD34 positive cells harvested from peripheral blood, umbilical cord blood or bone marrow may be differentiated into dendritic cells by adding to the culture medium combinations of GM-CSF, IL-3, TNFα, CD40 ligand, lipopolysaccharide LPS, flt3 ligand and/or other compound(s) that induce differentiation, maturation and proliferation of dendritic cells. Dendritic cells are conveniently categorized as “immature” and “mature” cells, which allows a simple way to discriminate between two well characterized phenotypes. However, this nomenclature should not be construed to exclude all possible intermediate stages of differentiation. Immature dendritic cells are characterized as APC with a high capacity for antigen uptake and processing, which correlates with the high expression of Fcy receptor and mannose receptor. The mature phenotype is typically characterized by a lower expression of these markers, but a high expression of cell surface molecules responsible for T cell activation such as class I and class II MHC, adhesion molecules (e.g., CD54 and CD11) and costimulatory molecules (e.g., CD40, CD80, CD86 and 4-1BB).

APCs may generally be transfected with a polynucleotide encoding the tumour protein (e.g. MAGE-3, Her2/neu, or derivative thereof) such that the tumour polypeptide, or an immunogenic portion thereof, is expressed on the cell surface. Such transfection may take place ex vivo, and a composition or vaccine comprising such transfected cells may then be used for therapeutic purposes, as described herein. Alternatively, a gene delivery vehicle that targets a dendritic or other antigen presenting cell may be administered to a patient, resulting in transfection that occurs in vivo. In vivo and ex vivo transfection of dendritic cells, for example, may generally be performed using any methods known in the art, such as those described in WO 97/24447, or the gene gun approach described by Mahvi D. M. et al., Immunology and Cell Biology, 1997, 75:456-460. Antigen loading of dendritic cells may be achieved by incubating dendritic cells or progenitor cells with the tumour polypeptide, DNA (naked or within a plasmid vector) or RNA; or with antigen-expressing recombinant bacterium or viruses (e.g., vaccinia, fowlpox, adenovirus or lentivirus vectors).

Other suitable delivery systems include microspheres wherein the antigenic material is incorporated into or conjugated to biodegradable polymers/microspheres so that the antigenic material can be mixed with a suitable pharmaceutical carrier and used as a vaccine. The term “microspheres” is generally employed to describe colloidal particles which are substantially spherical and have a diameter in the range 10 nm to 2 mm. Microspheres made from a very wide range of natural and synthetic polymers have found use in a variety of biomedical applications. This delivery system is especially advantageous for proteins having short half-lives in vivo requiring multiple treatments to provide efficacy, or being unstable in biological fluids or not fully absorbed from the gastrointestinal tract because of their relatively high molecular weights. Several polymers have been described as a matrix for protein release. Suitable polymers include gelatin, collagen, alginates, dextran. Delivery systems may include biodegradable poly(DL-lactic acid) (PLA), poly(lactide-co-glycolide) (PLG), poly(glycolic acid) (PGA), poly(E-caprolactone) (PCL), and copolymers poly(DL-lactic-co-glycolic acid) (PLGA). Other systems may include heterogeneous hydrogels such as poly(ether ester) multiblock copolymers, containing repeating blocks based on hydrophilic poly-(ethylene glycol) (PEG) and hydrophobic poly(butylene terephtalate) (PBT), or poly(ehtykene glycol)-terephtalate/poly(-butylene terephtalate) (PEGT/PBT) (Sohier et al. Eur. J. Pharm and Biopharm, 2003, 55, 221-228). Systems may provide a sustained release for 1 to 3 months such as PLGA, PLA and PEGT/PBT.

The treatment regime will be significantly varied depending upon the size and species of patient concerned, the amount of nucleic acid vaccine and/or protein composition administered, the route of administration, the potency and dose of any adjuvant compounds used and other factors which would be apparent to a skilled medical practitioner.

The invention will be further described by reference to the following, non-limiting, examples:

### EXAMPLE I

### Vaccine Preparation Using QS-21-Based Immunogenic Compositions

I.1.—Immunogenic Preparation Containing QS21 & 3 de-O-acylated Monophosphoryl Lipid A (3D-MPL) in an Oil in Water Emulsion (AS02 Formulation):

This adjuvant system AS02 has been previously described WO95/17210.

3D-MPL: is an immunostimulant derived from the lipopolysaccharide (LPS) of the Gram-negative bacterium Salmonella Minnesota. MPL has been deacylated and is lacking a phosphate group on the lipid A moiety. This chemical treatment dramatically reduces toxicity while preserving the immunostimulant properties (Ribi, 1986). Ribi Immunochemistry produces and supplies MPL to SB-Biologicals.

QS21: is a natural saponin molecule extracted from the bark of the South American tree Quillaja saponaria Molina. A purification technique developed to separate the individual saponins from the crude extracts of the bark, permitted the isolation of the particular saponin, QS21, which is a triterpene glycoside demonstrating stronger adjuvant activity and lower toxicity as compared with the parent component. QS21 has been shown to activate MHC class I restricted CTLs to several subunit Ags, as well as to stimulate Ag specific lymphocytic proliferation (Kensil, 1992). Aquila (formally Cambridge Biotech Corporation) produces and supplies QS21 to SB-Biologicals.

The oil/water emulsion is composed an organic phase made of of 2 oils (a tocopherol and squalene), and an aqueous phase of PBS containing Tween 80 as emulsifier. The emulsion comprised 5% squalene 5% tocopherol 0.4% Tween 80 and had an average particle size of 180 nm and is known as SB62 (see WO 95/17210).

Preparation of Emulsion SB62 (2 Fold Concentrate):

Tween 80 is dissolved in phosphate buffered saline (PBS) to give a 2% solution in the PBS. To provide 100 ml two fold concentrate emulsion 5 g of DL alpha tocopherol and 5 ml of squalene are vortexed to mix thoroughly. 90 ml of PBS/Tween solution is added and mixed thoroughly. The resulting emulsion is then passed through a syringe and finally microfluidised by using an M110S microfluidics machine. The resulting oil droplets have a size of approximately 180 nm.

Formulations:

A typical formulation containing 3D-MPL and QS21 in an oil/water emulsion is performed as follows: 20 μg-25 μg C-LytA P2-P501S are diluted in 10 fold concentrated of PBS pH 6.8 and H₂O before consecutive addition of SB62 (50 μl), MPL (20 μg), QS21 (20 μg), optionally comprising CpG oligonucleotide (100 μg) and 1 μg/ml thiomersal as preservative. The amount of each component may vary as necessary. All incubations are carried out at room temperature with agitation.

I.2.—Immunogenic Preparation Containing QS21 & CpG in a Liposomal Formulation (AS15 Adjuvant):

This adjuvant system AS15 has been previously described WO00/62800.

AS15 is a novel combination of the two adjuvant systems, AS01B and AS07A. AS01B is composed of liposomes containing 3D-MPL and QS21 and AS07A is composed of CpG 7909 (also known as CpG 2006) in phosphate buffer saline.

QS-21 is as described above.

CDG: CpG ODN 7909 is a synthetic single-stranded phosphorothioate oligodeoxy-nucleotide (ODN) of 24 bases length. Its base sequence, which is 5′-TCGTCGTTTTG-TCGTTTTGTCGTT-3′, has been optimised for stimulation of the human immune system. CpG DNA or synthetic ODN containing CpG motifs are known to activate dendritic cells, monocytes and macrophages to secrete TH1-like cytokines and to induce TH1 T cell responses including the generation of cytolytic T cells, stimulate NK cells to secrete IFNg and increase their lytic activity, they also activate B cells to proliferate (Krieg A et al. 1995 Nature 374: 546, Chu R et al. 1997 J. Exp. Med. 186: 1623). CpG 7909 is not antisense to any known sequence of the human genome. CpG 7909 is a proprietary adjuvant developed by and produced on behalf of Coley Pharmaceutical Group, Inc., MA, US.

Formulations with CPG:

Formulations were performed the days of injections. The volume of injection for one mouse was 50 or 100 μl. A typical formulation containing CpG, 3D-MPL and QS21 in liposomes is performed as follows: 20 μg-25 μg antigen was diluted with H₂O and PBS pH 7.4 for isotonicity. After 5 min., QS21 (0.5 μg) mixed with liposomes in a weight ratio QS21/cholesterol of 1/5 (referred to as DQ) was added to the formulation. 30 min later 10 μg of CpG (ODN 2006) was added 30 min prior addition of 1 μg/ml of thiomersal as preservative. All incubations are carried out at room temperature with agitation.

### EXAMPLE II

### Effect of mlL18 in Combination with HPV16 ProteinD-E7 Vaccine Adjuvanted with AS02 in the TC1 Therapeutic Model in E7-Tg Mice and non E7-Tg Mice

II.1. Experimental Design

7 groups of 5 female E7 Tg (C. Ledent et al. PNAS (USA) 1990, 87; 6176-6180) or non Tg C57BI/6 (Iffa Credo) mice received a tumour challenge of 10e6 TC1 cells (SC) in 200 μl at day 0.

Transgenic Mice Expressing HPV16 E7 Protein:

The transgenic mouse strain has been generated by M. Parmentier and C. Ledent at the IRIBHN (ULB). (Ref: PNAS (USA) 1990, 87; 6176-6180). As transgenic mice live with the E7 HPV16 gene from birth, they are considered “tolerant” to this gene: E7 from HPV 16, in this situation is considered as a “self antigen”. The expression of the transgene is driven by the thyroglobulin promoter. As Thyroglobulin is constitutively expressed only In the Thyroid, E7 is expressed in the thyroid. As a consequence of this expression, thyroid cells proliferate, mouse develop goiter and nodules which after 6 months to 1 year can evoluate in invasive cancer.

Tumour Cell Line TC1:

Primary lung epithelial cells from C57BL/6 mice were immortalised by HPV 16 E6 and E7 and then transformed with an activated ras oncogene, producing a tumourigenic cell line expressing E6 and E7 (Lin K Y et al. 1996). The E7 expression has been verified by FACS analysis of fixed and permeabilised TC1 cells using the mouse anti-HPV 16 E7 Mab (Triton Corp. Alameda, Calif.).

Vaccines: with 5 μg of PD1/3E7 (PDE7 batch 02/025) prepared as described in WO99/10375, adjuvanted in the QS-21 containing AS02B adjuvant (1/5^{th} of a human dose (MPL20 μg/ QS21 20 μg/SB62 50 μl) were given intra-muscularly (IM) at days 7 and 14.

Murine IL18—mlL-18 (batch_SB-528775 lot MJG-28800-176 at 1 mg/ml) was given S.C. in 100 μl, daily, for 3 weeks (starting at day 7)

II.2. In Vivo Tumour Growth:

TC1 cells growing in vitro culture were trypsinised, washed two times in serum-free medium and were injected S.C. in the right flank of the mice.

To assess treatment of established tumours, TC1 cells were injected at a dose of 1×10e6 cells/mouse. One and two weeks after the tumour cell injection, mice were vaccinated IM with 5 μg in 100 μl of protD 1/3 E7 His prepared as described in WO99/10375 and adjuvanted in the QS-21 containing AS02 adjuvant or with PBS alone. Five mice were used in each group.

Groups of Mice

gr1: PBS
gr2: PDE7 AS02B
gr3: PDE7 AS02B+100 μg mlL-18
gr4: PDE7 AS02B+1 μg mlL-18
gr5: PDE7 AS02B+0.1 μg mIL-18
gr6: 1 μg mlL18
gr7: 0.1 μg mlL18

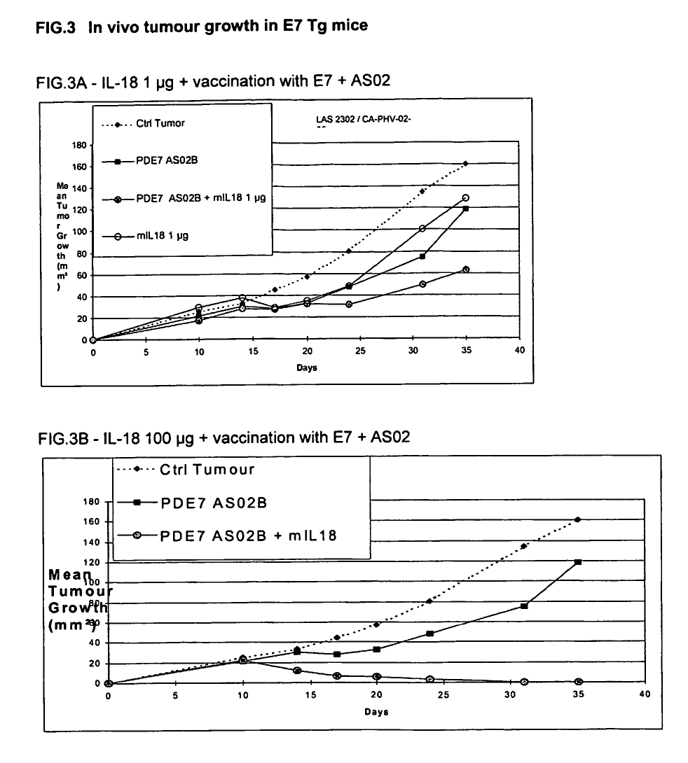

Mice were monitored twice a week for in vivo tumour growth during five weeks (until day 35). Serology (Ig tot and isotypes) was analysed at day 35. The mean tumour mass/group (expressed in mm2 for each group of 5 animals) is shown in FIG. 2 control non Tg mice) and in FIG. 3 (E7 Tg mice). FIG. 2A shows the results obtained with 1 μg of IL-18 and FIG. 2B shows the results obtained with 100 μg IL-18. FIG. 3A shows the results obtained with 1 μg of IL-18 and FIG. 3B shows the results obtained with 100 μg IL-18.

Table 1 below summarises the percentage of mice which completely reject their tumour upon vaccination in combination or not with repeated injections of IL-18 and also clearly show the benefit of combining vaccine and high dose of IL18.

TABLE 1

| | % Complete | |
|---|---|---|
| | regression | % Complete regression |
| Groups of mice | Control mice | E7Tg mice |
| PBS | 0 | 0 |
| PDE7 + AS02B | 20 | 0 |
| PDE7 + AS02B 0.1 μg IL18 | 20 | 0 |
| PDE7 + AS02B 1 μg IL18 | 0 | 0 |
| PDE7 + AS02B 100 μg IL18 | 100 | 100 |
| 0.1 μg mIL18 | 0 | 0 |
| 1 μg mIL18 | 0 | 0 |
| 100 μg mIL18 | 20 | 0 |
| (from previous exp.) | | |

II.3. Conclusion

The data obtained in this experiment demonstrate that:
There is a clear benefit to combine IL-18 with the E7 vaccine adjuvanted with QS21-containing AS02B on tumour regression both in control non transgenic as well as in E7Tg mice.
The only groups of mice which remains tumour-free after the challenge are the one receiving both the vaccine and the IL18
The benefit in combining is also dose dependent.
   II.4 Serology—Immunological Read-Outs

The antibody response and isotypic profile (on pooled sera) were measured by Elisa using PD1/3E7-16 (02/025) as coating antigen as described below.

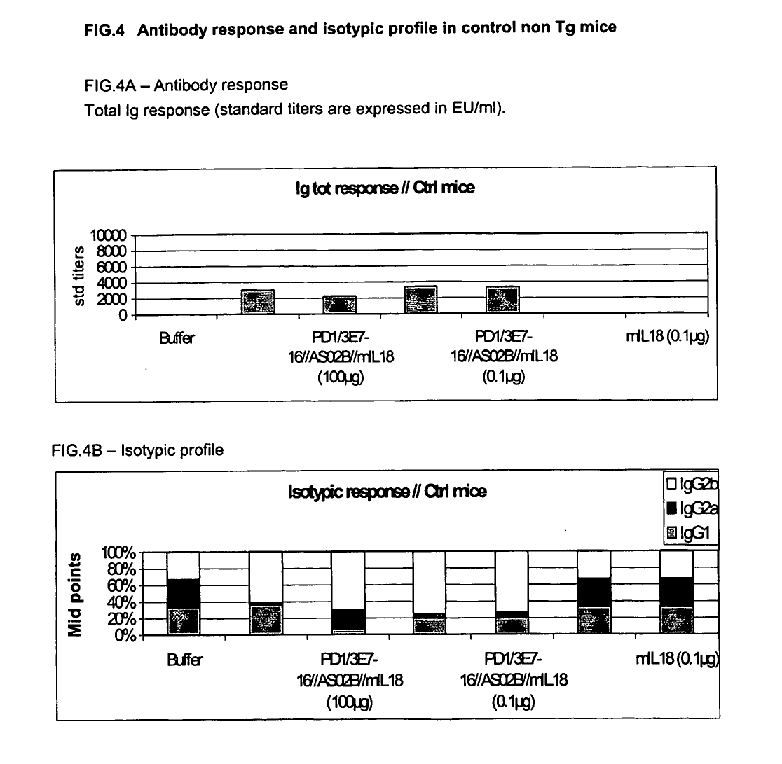

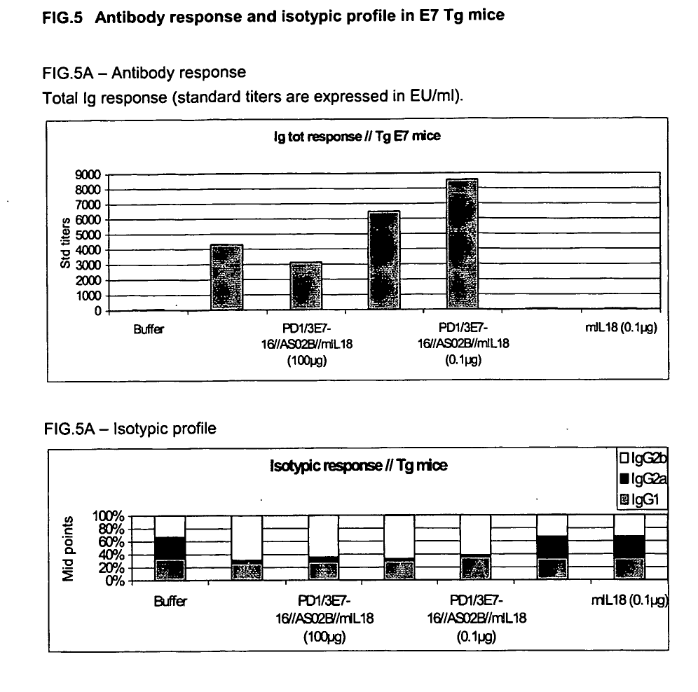

Individual serum were taken at the same time as the organs were taken and submitted to indirect ELISAs. 2 μg/ml of purified E7 protein was used as coated antigen. After saturation in PBS 0.1% tween 20 1% BSA 1 hour at 37° C., the sera were serially diluted (starting at 1/100) in the saturation buffer and incubated 90 min at 37° C. After washing in PBS Tween 20 0.1%, biotinylated goat Anti mouse Ig (1/5000) or goat anti-mouse Ig subclass (total IgG, IgG1, IgG2a, IgG2b) antisera (1/5000) were used as second antibodies, after an incubation of 90 min at 37° C., streptavidin coupled to peroxydase was added and TMB (tetra-methyl-benzidine/peroxide) was used as substrate. After 10 min, the reaction was stopped with H2SO4 0.5 M and the O.D.450 was determined. FIG. 4 shows the results obtained with control non Tg mice, while FIG. 5 shows the results obtained with E7 Tg mice.

II.5 Conclusion

In control mice receiving the IL-18 alone, no E7-specific antibodies are detected as expected. No major difference in total Ig response was seen whatever the group. The addition of IL-18 tends to improve the TH1 isotypic profile especially at high dose in combination with the vaccine

In E7Tg mice there seems to be an inverse relationship between the dose of IL-18 and the antibody level to E7. There is no major impact of the addition of IL-18 on the isotypic profile.

II.6 Overall Conclusion

IL-18 by itself impacts on TC1 tumour growth in a dose dependent manner;
There is a clear benefit on TC1 tumour growth to combine injection especially when high doses of mIL-18 (100 μg) are combined with E7+AS02B vaccination, in both control and E7Tg mice;
IL-18 slightly affects the antibody response induced by vaccination (better TH1 isotypic profile in control mice and better titers in E7Tg mice)

### EXAMPLE III

### Effect of mlL18 in Combination with Her2/neu Vaccine Adjuvanted with AS15 in the TC1 Her2 Therapeutic Model

III.1. Experimental Design

Vaccine

The Her-2/neu vaccine is ECD-PhD and comprises the entire extracellular domain (comprising amino acid 1-645) and an immunogenic portion of the intracellular domain comprising the phosphorylation domain. Such vaccine construct is disclosed in WO00/44899 and is called dHER2.

The dHER2 protein was co-lyophilised with CpG by diluting the antigen in a mix of H₂O, saccharose and NaH2PO4/K2HPO4. After 5 minutes, CpG ODN 7909 was added to obtain a final bulk containing 625 μg/ml of Her2neu, 1250 μg/ml of CpG, 3.15% saccharose and 5 mM PO4 pH 7 before freeze-drying. The final bulk was lyophilised according a 3 days cycle. For the extemporaneous formulation, the lyophilised cake containing CpG and antigen was resuspended with 625 μl of AS01B diluant containing 100 μg/ml of MPL and DQ.

Animals were injected with 50 ρl containing 25 ρg of Her2/neu, 50 μg of CpG and 5 μg of MPL and DQ.

Tumour Model Expressing HER-2/neu

The tumour model used in these experiment: TC1HER2 was generated by retroviral transduction of the TC1 cells (provided Dr T. C. Wu John's Hopkins University Baltimore) with a recombinant retoviruses encoding HER-2/neu).

Individual clones have been isolated, amplified and the stability of HER2/neu and MHC class I expression was confirmed by flow cytometry.

Groups of Mice:

4 groups of 5 female CB6F1 mice have received at day0 a sub-cutaneous (SC) challenge with 2×10e6 TC1 Her2 c18 cells followed by vaccination with either:
gr1: PBS
gr2: daily injection of 100 μg of mlL18 (murine) from day 7 to day 27 (SC)
gr3: 25 μg of dHER2 protein in AS15 at days 7 and 14 (IM)
gr4: the combination of the vaccine and the mlL18
   II.2. In Vivo Tumour Growth and Mortality:

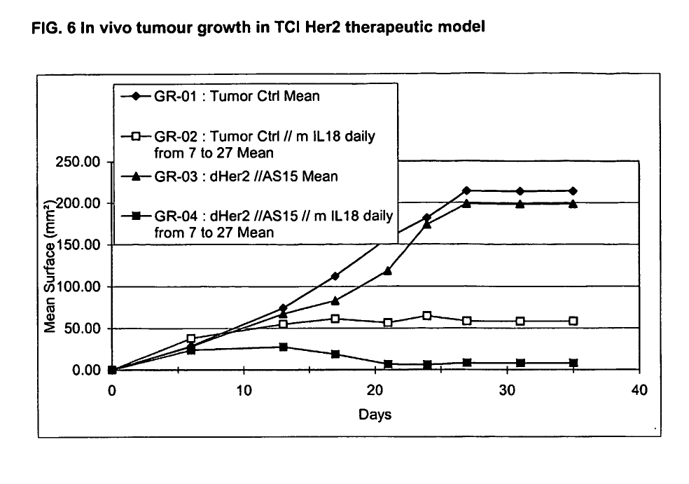

The results are shown in FIG. 6 and in Table 2.

TABLE 2
percentage of mice which remain tumour-free, 27 days
after the TC1HER2 tumour challenge.

| | |
|---|---|
| PBS | 0% |
| mIL18 | 20% (mortality: ⅖) |
| dHER2/AS15 | 0% |
| dHER2/AS15 | 60% (⅖ develop a little tumor) |
| mIL18 | |

II.3 Conclusion

A vaccine strategy based on the use of a recombinant purified HER-2/neu protein (dHER2) formulated in an adjuvant (AS15) combined with repeated injection of murine recombinant IL-18 did give improved results on pre-established tumours which express the HER2/neu antigen, as compared to a vaccination strategy with either the vaccine composition or the IL-18 alone. Vaccination based on the use of recombinant dHER protein formulated in the AS15 adjuvant had previously been shown to protect very efficiently mice against a challenge with these tumour cells expressing the HER2/neu antigen. This protection is specific for the HER2/neu antigen and is associated with the induction of a long term immune memory. In this more stringent therapeutic model were tumours are pre-established, vaccinations have been shown to be less effective having only a limited impact on the growing tumour (no mice completely reject tumors in these conditions). Surprisingly however, when both treatments were given concomittlantly, a synergy is observed and 60% of the mice remain completely tumour-free while 40% only develop a small tumour. In conclusion, there is a clear benefit to combine mlL-18 and the vaccines as shown in table 2. This could mean that both the induction of HER2/neu specific T cell responses by the vaccine and the activation of the immune system by repeated injection of IL-18 are crucial to get tumour regression.

### EXAMPLE IV

### Effect of mIL18 in Combination with MAGE-3 Vaccine Adjuvanted with ?? in the TC1 Mage3 Therapeutic Model

III.1. Experimental Design

Vaccine

A tumour model expressing the Mage3 tumour antigen has been generated (TC1Mage3) by genetically modifying the TC1 parental cells by classical transfection of a DNA plasmid coding for Mage3 (PcDNA3 Mage3). This tumour model was generated by transfecting the parental TC1 cells (provided by T. C. Wu at John's Hopkins University, Baltimore) with a PcDNA3 plasmid coding for Mage3. The transfection has been performed using lipofectamin according to the recommendation of the kit's provider (Gibco BRL Life Technologies, cat no 18324-012).

These cells are tumourigenic and 100% of the mice challenged with 2 10e6 TC1 Mage3 cells develop a tumour.

4 groups of 5 female C57BU6 mice will receive at day 0 a sub-cutaneous (SC) challenge with 2×10e6 TC1Mage3 cells followed by vaccinationn with either
gr1: PBS
gr2: daily injection of 100 μg of mlL18 (murine) from day 7 to day 27 (SC)
gr3: 10 μg of Mage3 protein in AS15 at days 7 and 14 (IM)
gr4: the combination of the vaccine and the mIL18

The ability of Mage3 in AS15 vaccination, IL18 injections and combined treatment to induce tumour regression is assessed. The impact of vaccination or/and IL18 treatment on immune parameter is also measured (lymphoproliferation, cytokine production . . . ).

1. A method of enhancing an immune response to an antigen in a mammal, comprising administering to the mammal a safe and effective amount of 1) an IL-18 polypeptide or bioactive fragment or variant thereof, and 2) an immunogenic composition comprising an antigen or immunogenic derivative thereof and a saponin adjuvant.

2. A method according to claim 2, wherein the antigen or immunogenic derivative thereof is derived from an organism selected from the group of: Human Immunodeficiency virus HIV-1, human herpes simplex viruses, cytomegalovirus, Rotavirus, Epstein Barr virus, Varicella Zoster Virus, from a hepatitis virus such as hepatitis B virus, hepatitis A virus, hepatitis C virus and hepatitis E virus, from Respiratory Syncytial virus, parainfluenza virus, measles virus, mumps virus, human papilloma viruses, flaviviruses or Influenza virus, from Neisseria spp, Moraxella spp, Bordetella spp; Mycobacterium spp., including M. tuberculosis; Escherichia spp, including enterotoxic E. coli; Salmonella spp,; Listeria spp; Helicobacter spp; Staphylococcus spp., including S. aureus, S. epidermidis; Borrelia spp; Chlamydia spp., including C. trachomatis, C. pneumoniae; Plasmodium spp., including P. falciparum; Toxoplasma spp., and Candida spp.

3. A method of reducing the severity of a cancer in a patient, comprising administering to a patient in need thereof a safe and effective amount of 1) an IL-18 polypeptide or bioactive fragment or variant thereof and 2) an immunogenic composition comprising a tumour-associated antigen or immunogenic derivative thereof and a saponin adjuvant.

4. A method according to claim 3, wherein the tumour-associated antigen or immunogenic derivative thereof is selected from the group of: an antigen from the MAGE family, PRAME, BAGE, LAGE 1, LAGE 2, SAGE, HAGE, XAGE, PSA, PAP, PSCA, prostein, P501S, HASH2, Cripto, B726, NY-BR1.1, P510, MUC-1, Prostase, STEAP, tyrosinase, telomerase, survivin, CASB616, P53, and her 2 neu.

5. The method according to claim 1, wherein the IL-18 polypeptide or bioactive fragment or variant thereof and the immunogenic composition are administered simultaneously, separately or sequentially in any order.

6. The method according to claim 5 wherein the IL-18 polypeptide or bioactive fragment or variant thereof and the immunogenic composition are administered simultaneously in the form of a combined pharmaceutical preparation.

7. The method according of claim 1, wherein the IL-18 polypeptide or bioactive fragment or variant thereof is from human or murine origin.

8. The method according to claim 7, wherein IL-18 is:the polypeptide of SEQ ID NO:6 or SEQ ID NO:7 or bioactive fragment or derivative thereof.

9. The method according to claim 1, wherein the saponin adjuvant is chosen from the group of: QS-21 and QS-17.

10. A combined preparation comprising as active ingredients the following individual components: (1) IL-18 polypeptide or bioactive fragment or variant thereof and (2) immunogenic composition comprising an antigen and a saponin adjuvant, the active ingredients being for the simultaneous, separate or sequential use for the prophylaxis and/or treatment of infectious diseases, cancer, autoimmune diseases and related conditions.

11. The combined preparation according to claim 10, wherein components (1) and (2) are admixed in a composition.

12. The combined preparation according to claim 10, wherein the immunogenic composition comprises a tumour-associated antigen or immunogenic derivative thereof and is prophylactically or therapeutically active against cancer.

13. The combined preparation according to claim 12, wherein the tumour-associated antigen or immunogenic derivative thereof is selected from the group of: an antigen from the MAGE family, PRAME, BAGE, LAGE 1, LAGE 2, SAGE, HAGE, XAGE, PSA, PAP, PSCA, prostein, P501S, HASH2, Cripto, B726, NY-BR1.1, P510, MUC-1, Prostase, STEAP, tyrosinase, telomerase, survivin, CASB616, P53, and her 2 neu.

14. The combined preparation according claim 10, wherein the IL-18 polypeptide or bioactive fragment or variant thereof is from human or murine origin.

15. The combined preparation according to claim 14, wherein IL-18 is the polypeptide of SEQ ID NO:6 or SEQ ID NO:7 or bioactive fragment or derivative thereof.

16. The combined preparation according claim 10, wherein the saponin adjuvant is chosen from the group of: QS-21 and QS-17.

17. The combined preparation as claimed in claim 10, wherein the immunogenic composition additionally comprises an immunostimulant chemical selected from the group of: 3D-MPL, cholesterol, CpG oligonucleotide containing at least one immunostimulatory CG dinucleotide, aluminium hydroxide, aluminium phosphate, and tocopherol, and an oil in water emulsion or a combination of two or more of the said adjuvants.

18. The combined preparation as claimed in claim 17, wherein the immunogenic composition adjuvant comprises 3D-MPL, QS21, cholesterol, an oil in water emulsion.

19. The combined preparation as claimed in claim 18, wherein the oil in water emulsion comprises squalene, tocopherol, and polyoxyethylenesorbitan monooleate (Tween 80).

20. The combined preparation as claimed in claim 17, wherein the immunogenic composition comprises QS21, cholesterol, and a CpG oligonucleotide containing at least one immunostimulatory CG dinucleotide.

21. The combined preparation as claimed in claim 10, wherein both active components are in the form of injectable solutions.

22. A pharmaceutical kit comprising as active ingredients: (1) an IL-18 polypeptide or bioactive fragment thereof; and (2) an immunogenic composition comprising an antigen or immunogenic derivative thereof and a saponin adjuvant, the active ingredients being for the simultaneous, separate or sequential use for the prophylaxis and/or treatment of infectious diseases, cancer, and auto-immune diseases.

23. The pharmaceutical kit according to claim 22, wherein the immunogenic composition comprises a tumour-associated antigen or immunogenic derivative thereof and is prophylactically or therapeutically active against cancer.

24. The pharmaceutical kit according to claim 23, wherein the tumour-associated antigen or immunogenic derivative thereof is selected from the group of: an antigen from the MAGE family, PRAME, BAGE, LAGE 1, LAGE 2, SAGE, HAGE, XAGE, PSA, PAP, PSCA, prostein, P501S, HASH2, Cripto, B726, NY-BR1.1, P510, MUC-1, Prostase, STEAP, tyrosinase, telomerase, survivin, CASB616, P53, and her 2 neu.

25. The combined preparation as claimed in claim 10 for use in medicine.

26. The method as claimed in of claim 1, which comprises the use of a combined preparation according to claim 10.

27.-32. (canceled)

33. A method for the prophylaxis and/or treatment of a patient suffering from or susceptible to infectious diseases, cancer, autoimmune diseases and related conditions, and already primed with an immunogenic composition, comprising an antigen or immunogenic derivative thereof and a saponin adjuvant, comprising administering to the patient a safe and effective amount of an IL-18 polypeptide or bioactive fragment or variant thereof.

34. A method for the prophylaxis and/or treatment of a patient suffering from or susceptible to infectious diseases, cancer, autoimmune diseases and related conditions, and already primed with an immunogenic composition, comprising an antigen or immunogenic derivative thereof and a saponin adjuvant, comprising administering to the patient a safe and effective amount of an IL-18 polypeptide or bioactive fragment or variant thereof.

35. The method according to claims 33, wherein the antigen is a tumour-associated antigen, and the cancer is selected from the group of: breast cancer, lung cancer, NSCLC, colon cancer, melanoma, ovarian cancer, bladder cancer, head and neck squanmous carcinoma, and esophageal cancer.

36. The method according to claim 33, wherein the IL-18 polypeptide or bioactive fragment or variant thereof is from human or murine origin.

37. The method according to claim 36, wherein IL-18 is the polypeptide of SEQ ID NO.6 or SEQ ID NO.7 or bioactive fragment or derivative thereof.

38. The method according to any claim 33, wherein the saponin adjuvant is chosen from the group of: QS-21 and QS-17.
